(19) United States
(12) Patent Application Publication
(10) Pub. No.: US 2017/0327834 A1
(43) Pub. Date: Nov. 16, 2017

(54) PESTICIDAL MICRORNA CARRIERS AND USE THEREOF

(71) Applicant: SYNGENTA PARTICIPATIONS AG, Basel (CH)

(72) Inventors: Guo-Qing Tang, Durham, NC (US); Xiang Huang, Cary, NC (US)

(73) Assignee: SYNGENTA PARTICIPATIONS AG, Basel (CH)

(21) Appl. No.: 15/528,540

(22) PCT Filed: Dec. 15, 2015
(86) PCT No.: PCT/US2015/065804

§ 371 Date: May 22, 2017

Related U.S. Application Data
(60) Provisional application No. 62/091,810, filed on Dec. 15, 2014.

(51) Int. Cl. C12N 15/82

(52) U.S. Cl. CPC C12N 15/8218; C12N 15/8286; C12N 15/8279; C12N 15/8282; C12N 15/8285

(57) ABSTRACT

The invention relates to synthetic plant miRNA precursor molecules that are resistant to processing in plants but functional in plant pests. The invention further relates to methods for using the synthetic plant miRNA precursor molecules to protect plants against pests.

### STATEMENT OF PRIORITY

This application claims the benefit, under 35 U.S.C. §119(e), of U.S. Provisional Application No. 62/091,810, filed Dec. 15, 2014, the entire contents of which is incorporated herein.

### STATEMENT REGARDING ELECTRONIC FILING OF A SEQUENCE LISTING

A Sequence Listing in ASCII text format, submitted under 37 C.F.R. §1.821, entitled 80554-WO-REG-ORG-P-1.txt, 6,504 bytes in size, generated on, and filed via EFS-Web, is provide in lieu of a paper copy. This Sequence Listing is hereby incorporated by reference into the specification for its disclosures.

### FIELD OF THE INVENTION

The invention relates to synthetic plant miRNA precursor molecules that are resistant to processing in plants but functional in plant pests. The invention further relates to methods for using the synthetic plant miRNA precursor molecules to protect plants against pests.

### BACKGROUND

MicroRNAs (miRNAs) are non-protein coding RNAs, generally of between about 17 to about 25 nucleotides (commonly about 20-24 nucleotides in plants). miRNAs direct cleavage in trans of target transcripts, regulating the expression of genes involved in various pathways (Bartel, Cell, 116:281-297 (2004); Zhang et al., Dev. Biol. 289:3-16 (2006)). miRNAs have been shown to be involved in different aspects of plant growth and development as well as in signal transduction and protein degradation. In addition, growing evidence indicates that small endogenous RNAs including miRNAs may also be involved in biotic stress responses such as parasite attack. Since the first miRNAs were discovered in plants (Reinhart et al., Genes Dev. 16:1616-1626 (2002), Park et al., Curr. Biol. 12:1484-1495 (2002)), many hundreds have been identified. Further, many plant miRNAs have been shown to be highly conserved across very divergent taxa. (Floyd et al., Nature 428:485-486 (2004); Zhang et al., Plant 46:243-259 (2006)). Many microRNA genes (MIR genes) have been identified and made publicly available in a database (“miRBase,” microrna.sanger.ac.uk/sequences). miRNAs are also described in U.S. Patent Publications 2005/0120415 and 2005/144669A1, the entire contents of which are incorporated by reference herein.

Genes encoding miRNAs yield primary miRNAs (“pri-miRNA”) of 70 to 300 bp in length that can form imperfect stem-loop structures. A single pri-miRNA may contain from one to several miRNA precursors. In animals, pri-miRNAs are processed in the nucleus into shorter hairpin RNAs of about 65 nucleotides (referred to as precursor miRNAs (pre-miRNAs)) by the RNaseIII enzyme Drosha and its cofactor DGCR8/Pasha. The pre-miRNA is then exported to the cytoplasm, where it is further processed by another RNaseIII enzyme, Dicer, releasing a miRNA (guide strand)/miRNA* (passenger or carrier strand) duplex of about 22 nt in size. In contrast to animals, in plants, the processing of pri-miRNAs into mature miRNAs occurs entirely in the nucleus using a single RNaseIII enzyme, DCL-1 (Dicer-like 1). (Zhu, Proc. Natl. Acad. Sci. 105:9851-9852 (2008)). Many reviews on microRNA biogenesis and function are available, for example, see, Bartel, Cell 116:281-297 (2004), Murchison et al., Curr. Opin. Cell Biol. 16:223-229 (2004), Dugas et al., Curr. Opin. Plant Biol. 7:512-520 (2004) and Kim, Nature Rev. Mol. Cell Biol. 6:376-385 (2005).

One pathway for modifying crop plants to confer resistance to pests is to use RNA interference (RNAi). Currently, RNAi delivery approaches to confer resistance use either long hairpin RNAi (hpRNA) or artificial miRNA RNAi (amiRNA). However, the RNAi effect is not ideal because of the robust processing of these transcripts by endogenous DCL-4 and DCL-2 (targeting hpRNA), and DCL-1 (targeting amiRNA) in plant cells, resulting in a significantly reduced effective dose of RNAi molecules prior to being taken up by pests. The dose of the RNAi trigger is crucial to the success of targeted RNAi (Tomizawa et al., Appl. Entomol. Zool. 48:553-559 (2013)). The present invention addresses shortcomings in the art by providing RNAi molecules that are resistant to DCL-1 processing in plant cells but are competent to be processed in pest cells after uptake.

### SUMMARY OF THE INVENTION

The present invention is based, in part, on the development of a design algorithm for synthetic DCL-1 resistant miRNA precursors. The precursors of the invention provide a scaffold into which any guide strand can be placed for expression, e.g., a guide strand that targets a nucleic acid of interest in a plant pest.

One aspect of the invention relates to a synthetic plant microRNA (miRNA) precursor comprising four DCL-1 cleavage sites, the precursor comprising the following structures in loop-to-base order:

A. a terminal loop;

B. a neck stem;

C. a pair of mismatches and the flanking nucleotides surrounding a first cleavage site;

D. a pair of mismatches and/or bulges and the flanking nucleotides surrounding a second cleavage site; and/or

E. a series of mismatches and/or bulges and the flanking nucleotides between the second cleavage site and a third cleavage site;

wherein the precursor is modified at one or more of structures A, B, C, D, and E to be resistant to cleavage by a plant Dicer-like protein-1 (DCL-1) but susceptible to cleavage by Dicer or a Dicer-like protein of a plant pest.

An additional aspect of the invention relates to a method of producing a plant that is resistant to a plant pest, comprising introducing into a plant or plant part the synthetic plant miRNA precursor molecule, recombinant nucleic acid, expression cassette, or vector of the invention, thereby producing a transgenic plant or plant part that is resistant to a plant pest.

A further aspect of the invention relates to a method of producing a plant that is resistant to a plant pest, comprising introducing into a plant cell the synthetic plant miRNA precursor molecule, recombinant nucleic acid, expression cassette, or vector of the invention, and regenerating a plant or plant part from said plant cell, thereby producing a transgenic plant or plant part that is resistant to a plant pest.

Another aspect of the invention relates to a method of modulating the expression of a target polynucleotide or a target gene in a plant pest, comprising providing a plant produced by the methods of the invention; exposing the plant to the plant pest under conditions wherein the plant pest takes up the synthetic plant miRNA precursor; thereby modulating the expression of a target polynucleotide or a target gene in the plant pest.

An additional aspect of the invention relates to a method of controlling a plant pest, comprising providing a plant produced by the methods of the invention; exposing the plant to the plant pest under conditions wherein the plant pest takes up the synthetic plant miRNA precursor; thereby controlling the plant pest.

Another aspect of the invention relates to a method of reducing damage in a plant caused by a plant pest, the method comprising planting the seed of the invention, thereby reducing damage caused by the pest to a plant grown from the seed.

A further aspect of the invention relates to a method of providing a farmer with a means of controlling a plant pest, the method comprising supplying to the farmer the plant or the seed of the invention.

An additional aspect of the invention relates to a method of reducing damage in a plant caused by a plant pest, the method comprising applying to a plant or plant part the synthetic plant miRNA precursor molecule of the invention, thereby reducing damage caused by the pest.

Another aspect of the invention relates to a recombinant nucleic acid molecule, expression cassette, or vector comprising a nucleotide sequence encoding the synthetic plant miRNA precursor of the invention.

A further aspect of the invention relates to a composition comprising the synthetic plant miRNA precursor, recombinant nucleic acid molecule, expression cassette, or vector of the invention.

An additional aspect of the invention relates to plants, plant parts and cells comprising a synthetic DCL-1 resistant miRNA precursor of the invention as well as seeds, crops, harvested products and post harvest products produced from the plants, plant parts, and/or crops of the invention.

These and other aspects of the invention are set forth in more detail in the description of the invention below.

### BRIEF DESCRIPTION OF THE FIGURES

FIG. 1 shows a symbolic diagram of physical parameters for designing synthetic precursors defective to DCL-1 recognition and processing. The miRNA/miRNA* duplex is indicated in pink. The dotted lines are indications of changes and modifications.

FIG. 2 shows a schematic diagram of the targeted structural areas in which sequence contexts were altered. The miRNA/miRNA* duplex is indicated in pink. The dotted lines are indications of changes and modifications.

FIG. 3 is an illustration of the technical requirements for the design of DCL-1 resistant precursors based on the loop to base processing mechanism. Five important regions and structural features were subjected to changes or modifications.

FIGS. 10A-10C show a schematic representation of DCL-1 resistant precursor RNA folding energy correlated with terminal loop sizes. A. Mini-loop based precursor folding energy. B. Large loop based folding energy. C. Statistical calculations of folding energy.

FIG. 11 shows a schematic representation of an example illustrating the design of DCL-1 resistant synthetic precursor dp0017. Individual changes and modifications were shown in parallel with the blueprint of the structural backbone proposed in FIG. 2. The designed critical regions corresponding to the general structural skeleton are plotted with dash lines in blue.

FIG. 13 shows amplification of the full length synthetic precursor RNA by qRT-PCR. The synthetic DCL-1 preferred precursor dp0019 was used as the negative control.

FIG. 14 shows the DCL-1 resistant synthetic precursors that were selected for normal phenotype. The qRT-PCR reads of accumulation of these full length precursor RNAs was plotted against the negative control DCL-1 preferred synthetic precursor dp0019.

FIG. 15 depicts mortality in the western corn rootworm bioassay resulting from His4 siRNA inserted in precursor dp005. The assay time span was 16 days. The dose used in the assay was 100 ng/cm² as indicated in the figure.

FIG. 16 shows results from qRT-PCT assays from body sRNA analysis of insects that have ingested DCL-1 resistant synthetic precursors.

FIG. 17 shows results from qRT-PCT assays from frass sRNA analysis of insects that have ingested DCL-1 resistant synthetic precursors.

FIG. 18 shows the stability of DCL-1 resistant synthetic precursors in western corn rootworm gut juice+hemolymph and in whole body juice.

### DETAILED DESCRIPTION OF THE INVENTION

This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications and patent applications mentioned in this specification are indicative of the level of skill of those skilled in the art that this invention pertains. Further, publications, patent applications, patents, and other references mentioned herein are incorporated by reference in their entireties for the teachings relevant to the sentence and/or paragraph in which the reference is presented.

As used in the description of the embodiments of the invention and the appended claims, the singular forms “a,” “an,” and “the” are intended to include the plural forms as well, unless the context clearly indicates otherwise.

As used herein, “and/or” refers to and encompasses any and all possible combinations of one or more of the associated listed items.

The term “about,” as used herein when referring to a measurable value such as an amount of a compound, dose, time, temperature, and the like, refers to variations of 20%, 10%, 5%, 1%, 0.5%, or even 0.1% of the specified amount.

The terms “comprise,” “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the transitional phrase “consisting essentially of” (and grammatical variants) means that the scope of a claim is to be interpreted to encompass the specified materials or steps recited in the claim and those that do not materially alter the basic and novel characteristic(s)” of the claimed invention. Thus, the term “consisting essentially of” when used in a claim of this invention is not intended to be interpreted to be equivalent to “comprising.”

With respect to a polynucleotide sequence of this invention, the term “consists essentially of” (and grammatical variants) means a polynucleotide that consists of both the recited sequence (e.g., SEQ ID NO) and a total of ten or less (e.g., 1, 2, 3, 4, 5, 6, 7, 8, 9, or 10) additional nucleotides on the 5′ and/or 3′ ends of the recited sequence such that the function of the polynucleotide is not materially altered. The total of ten or less additional nucleotides includes the total number of additional nucleotides on both ends added together. The term “materially altered,” as applied to polynucleotides of the invention, refers to an increase or decrease in ability to express the encoded miRNA of at least about 50% or more or modulate the expression of a target polynucleotide or gene as compared to the expression level of a polynucleotide consisting of the recited sequence.

It will be understood that, although the terms “first,” “second,” etc. may be used herein to describe various elements, these elements should not be limited by these terms. These terms are only used to distinguish one element from another. Thus, a “first” element (e.g., a first promoter sequence) as described herein could also be termed a “second” element (e.g., a second promoter sequence) without departing from the teachings of the present invention.

As used herein, the term “double strand” can mean 100% complementarity or less than 100% complementarity between the two strands of the double strand (e.g., about 60%, 65%, 70%, 75%, 80%, 85%, 90%, 95%, 99%, 100% complementarity, or any range or value therein).

As used herein, with respect to nucleic acids, the term “exogenous” refers to a nucleic acid molecule that is not in the natural genetic background of the cell/organism in which it resides. In some embodiments, the exogenous nucleic acid molecule comprises one or more nucleotide sequences that are not found in the natural genetic background of the cell/organism. In some embodiments, the exogenous nucleic acid molecule can comprise one or more additional copies of a nucleotide sequence that is/are endogenous to the cell/organism.

As used herein, the terms “express,” “expresses,” “expressed” or “expression,” and the like, with respect to a nucleic acid molecule and/or a nucleotide sequence (e.g., RNA or DNA) indicates that the nucleic acid molecule and/or a nucleotide sequence is transcribed and, optionally, translated. Thus, a nucleic acid molecule and/or a nucleotide sequence may express a polypeptide of interest or, for example, a functional untranslated RNA.

As used herein, the term “heterologous” refers to a nucleotide/polypeptide that originates from a foreign species, or, if from the same species, is substantially modified from its native form in composition and/or genomic locus by deliberate human intervention.

The terms “increase,” “increasing,” “increased,” “enhance,” “enhanced,” “enhancing,” and “enhancement” (and grammatical variations thereof), as used herein, describe an elevation in the expression of a target gene or target polynucleotide (e.g., an elevation of at least about 20%, 25%, 30%, 35%, 40%, 45%, 50%, 75%, 100%, 125%, 150%, 175%, 200%, 350%, 300%, 350%, 400%, 450%, 500% or more). This increase in expression can be observed by comparing the expression of the target gene or target polynucleotide in a plant pest that has taken up a synthetic precursor molecule of the invention comprising a guide miRNA complementary to the target gene or target polynucleotide to expression of said target gene or target polynucleotide in a control plant pest that, for example, that has not taken up said synthetic precursor molecule of the invention comprising a guide miRNA complementary to the same target gene or target polynucleotide.

As used herein, the terms “reduce,” “reduced,” “reducing,” “reduction,” “diminish,” “suppress,” and “decrease” (and grammatical variations thereof), describe, for example, a decrease in the expression of a target gene or target polynucleotide as compared to a control as described herein. This decrease in expression can be observed by comparing the expression of the target gene or target polynucleotide in a plant pest that has taken up a synthetic precursor molecule of the invention comprising a guide miRNA complementary to the target gene or target polynucleotide to the expression of said target gene or target polynucleotide in a control plant pest that, for example, has not taken up said synthetic precursor molecule of the invention comprising a guide miRNA complementary to the same target gene or target polynucleotide.

As used herein, the terms “modulating,” “modulate,” “modulates” or grammatical variations thereof, means an alteration in the expression of a target gene or target polynucleotide by increasing or reducing the expression of said target polynucleotide or target gene.

In some embodiments, the recombinant nucleic acid molecules, and/or nucleotide sequences of the invention are “isolated.” An “isolated” nucleic acid molecule, an “isolated” nucleotide sequence or an “isolated” polypeptide is a nucleic acid molecule, nucleotide sequence or polypeptide that, by the hand of man, exists apart from its native environment and is therefore not a product of nature (i.e., non-naturally occurring). An isolated nucleic acid molecule, nucleotide sequence or polypeptide may exist in a purified form that is at least partially separated from at least some of the other components of the naturally occurring organism or virus, for example, the cell or viral structural components or other polypeptides or nucleic acids commonly found associated with the polynucleotide. In representative embodiments, the isolated nucleic acid molecule, the isolated nucleotide sequence and/or the isolated polypeptide is at least about 1%, 5%, 10%, 20%, 30%, 40%, 50%, 60%, 70%, 80%, 90%, 95%, or more pure.

In other embodiments, an isolated nucleic acid molecule, nucleotide sequence or polypeptide may exist in a non-native environment such as, for example, a recombinant host cell. Thus, for example, with respect to nucleotide sequences, the term “isolated” means that it is separated from the chromosome and/or cell in which it naturally occurs. A polynucleotide is also isolated if it is separated from the chromosome and/or cell in which it naturally occurs in and is then inserted into a genetic context, a chromosome and/or a cell in which it does not naturally occur (e.g., a different host cell, different regulatory sequences, and/or different position in the genome than as found in nature). Accordingly, the recombinant nucleic acid molecules, nucleotide sequences and their encoded functional nucleic acids or polypeptides are “isolated” in that, by the hand of man, they exist apart from their native environment and therefore are not products of nature, however, in some embodiments, they can be introduced into and exist in a recombinant host cell.

A “native” or “wild type” nucleic acid, nucleotide sequence, polypeptide or amino acid sequence refers to a naturally occurring or endogenous nucleic acid, nucleotide sequence, polypeptide or amino acid sequence. Thus, for example, a “wild type mRNA” is an mRNA that is naturally occurring in or endogenous to the organism. A “homologous” nucleic acid sequence is a nucleotide sequence naturally associated with a host cell into which it is introduced.

Also as used herein, the terms “nucleic acid,” “nucleic acid molecule,” “nucleotide sequence” and “polynucleotide” can be used interchangeably and encompass both RNA and DNA, including cDNA, genomic DNA, mRNA, synthetic (e.g., chemically synthesized) DNA or RNA and chimeras of RNA and DNA. The term polynucleotide, nucleotide sequence, or nucleic acid refers to a chain of nucleotides without regard to length of the chain. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases. The present invention further provides a nucleic acid that is the complement (which can be either a full complement or a partial complement) of a nucleic acid, nucleotide sequence, or polynucleotide of this invention.

Nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§1.821-1.825 and the World Intellectual Property Organization (WIPO) Standard ST.25.

As used herein, the term “substantially complementary” (and similar terms) means that two nucleic acid sequences are at least about 70%, 75%, 80%, 85%, 90%, 95%, 96%, 97%, 98%, 99% or more complementary. Alternatively, the term “substantially complementary” (and similar terms) can mean that two nucleic acid sequences can hybridize together under high stringency conditions (as described herein). Thus, in some embodiments, “substantially complementary” means about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% complementary, or any value or range therein).

The phrase “hybridizing specifically to” (and similar terms) refers to the binding, duplexing, or hybridizing of a molecule to a particular nucleic acid target sequence under stringent conditions when that sequence is present in a complex mixture (e.g., total cellular DNA or RNA) to the substantial exclusion of non-target nucleic acids, or even with no detectable binding, duplexing or hybridizing to non-target sequences. Selectively hybridizing sequences typically are at least about 40% complementary and are optionally substantially complementary or even completely complementary (i.e., 100% identical) to a target nucleic acid target sequence.

The term “bind(s) substantially” (and similar terms) as used herein refers to complementary hybridization between a nucleic acid molecule and a target nucleic acid and embraces minor mismatches that can be accommodated by reducing the stringency of the hybridization media to achieve the desired detection of the target nucleic acid sequence.

The terms “gene silencing,” “gene knockdown,” “reduction of gene expression,” “inhibition of gene expression,” “gene downregulation,” and “gene suppression” are used interchangeably to generally describe reductions of the amount of RNA transcribed from the gene and/or, in the case of a protein-encoding gene, protein translated from the transcribed mRNA. The transcribed RNA may be non-coding or protein-encoding. The term “non-coding” refers to polynucleotides that do not encode part or all of an expressed protein. Non-coding sequences include but are not limited to introns, enhancers, promoter regions, 3′ untranslated regions, 5′ untranslated regions, intergenic regions, and coding regions in the antisense direction. Measurement of transcribed RNA or translated protein can be done by using molecular techniques such as RNA solution hybridization, nuclease protection, Northern hybridization, reverse transcription, gene expression monitoring with a microarray, antibody binding, enzyme-linked immunosorbent assay (ELISA), Western blotting, radioimmunoassay (MA), other immunoassays, or fluorescence-activated cell analysis (FACS). Gene suppression can be the result of co-suppression, anti-sense suppression, transcriptional gene silencing, post-transcriptional gene silencing, or translational gene silencing. A “silenced,” “knocked-down,” “reduced,” “inhibited,” “down regulated,” or “suppressed” gene refers to a gene that is subject to silencing. “Target gene” or “target polynucleotide” is thus the gene or polynucleotide which is to be silenced. Gene silencing is “specific” for a target gene when silencing of the target gene occurs without manifest effects on other genes.

“RNA interference” or “RNAi” refers to sequence-specific or gene-specific suppression of gene expression that is mediated by interfering RNA.

“Interfering RNA” is RNA capable of causing gene silencing. Interfering RNA encompasses any type of RNA molecule capable of down-regulating or silencing expression of a target gene, including but not limited to sense RNA, antisense RNA, short interfering RNA (siRNA), microRNA (miRNA), double-stranded RNA (dsRNA), hairpin RNA (RNA) and the like. Methods to assay for functional interfering RNA molecules are well-known in the art.

The phrases “target-specific small interfering RNAs,” “target-specific siRNAs,” “target-specific microRNAs,” “target-specific miRNAs,” “target-specific amiRNAs,” and “target-specific nucleotide sequences” refer to interfering RNAs that have been designed to selectively or preferentially hybridize with nucleic acids in a target organism (e.g., target nucleic acid), such as a host organism (the organism expressing the target specific miRNA) or a consumer of the host organism.

Interfering RNA may be in the form of short double-stranded RNA (dsRNA) molecules like micro RNA (miRNA).

A dsRNA molecule need not be completely double-stranded, but comprises at least one double-stranded region comprising at least one functional double-stranded silencing element.

It is to be understood that the strands forming the at least one double-stranded region need not be 100% complementary. Strands having insertions, deletions, and single point mutations relative to each other are still capable of forming a double-stranded region. Thus, the strands of the at least one double-stranded region of a dsRNA molecule can be at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% complementary to each other, or any value or range therein.

It is also to be understood that the strands forming the at least one double-stranded silencing element (e.g., miRNA passenger strand/miRNA guide strand) need not be 100% complementary. Strands having insertions, deletions, and single point mutations relative to each other are still capable of forming a double-stranded silencing element. Thus, the strands of an at least one double-stranded silencing element of a dsRNA molecule can be at least about 70%, 75%, 80%, 85%, 90%, 95%, 99%, or 100% complementary to each other. In representative embodiments, a guide strand (targeting strand) and a passenger strand of a synthetic DCL-1 resistant miRNA precursor of the invention can be at least about 70% to about 90% (e.g., about 70%, 71%, 72%, 73%, 74%, 75%, 76%, 77%, 78%, 79%, 80%, 81%, 82%, 83%, 84%, 85%, 86%, 87%, 88%, 89%, 90%) complementary to each other, or any value or range therein.

In some aspects, a dsRNA may be a single strand that is capable of folding back on itself to form a hairpin RNA (hpRNA) or stem-loop structure. In the case of a hpRNA, the double-stranded region or ‘stem’ is formed from two regions or segments of the RNA that are essentially inverted complements of one another and possess sufficient complementarity to allow the formation of a double-stranded region. At least one functional double-stranded silencing element is present in this double-stranded region or ‘stem’ of the molecule. The stem-forming single-stranded regions are typically separated by a region or segment of the RNA known as the ‘loop’ region. This region can comprise any nucleotide sequence conferring enough flexibility to allow self-pairing to occur between the flanking complementary regions of the RNA. In general, the loop region is substantially single-stranded and acts as a spacer element between the inverted complements. In some representative embodiments, further loops and double stranded regions can be comprised within a larger loop region.

To “control” an organism (e.g., plant pest) means to inhibit, through a toxic effect, the ability of an organism (e.g., plant pest) to survive, grow, feed, and/or reproduce, or to limit damage or loss in crop plants that is related to the activity of the organism. To “control” an organism may or may not mean killing the organism, although it preferably means killing the organism.

“Resistant,” with respect to a plant being resistant to a plant pest, means that the plant incurs a reduced level of damage when exposed to the plant pest. The resistance can be partial, e.g., the level of damage is reduced by at least 20%, e.g., at least 30%, 40%, 50%, 60%, 70%, 80% or more compared to a plant that does not comprise a synthetic plant miRNA precursor of the invention.

“Pesticidally effective amount” or “effective pest controlling amount,” means the concentration or amount of a synthetic plant miRNA precursor of the invention that inhibits, through a toxic effect, the ability of pests to survive, grow, feed and/or reproduce, or to limit pest-related damage or loss in plants. “Pesticidally effective amount” or “effective pest controlling amount” may or may not mean killing the pests, although it preferably means killing the pests.

“Resistant to cleavage,” as applied to a synthetic plant miRNA precursor of the invention, means the precursor is not properly processed by DCL-1 to produce a mature miRNA. Resistance can be complete (e.g., 100% decrease in processing) or partial (e.g., a decrease in processing of at least 30%, 40%, 50%, 60%, 70%, 80% or more relative to a precursor that is not resistant to cleavage).

“Susceptible to cleavage,” as applied to a synthetic plant miRNA precursor of the invention, means the precursor is properly processed by Dicer or a Dicer-like protein of a plant pest to produce a mature miRNA. Susceptibility can be complete (e.g., 100% processing) or partial (e.g., processing at a level of at least 30%, 40%, 50%, 60%, 70%, 80% or more relative to an endogenous miRNA precursor of the plant pest).

The term “takes up” means that the plant pest ingests, incorporates, absorbs, or otherwise takes in the synthetic plant miRNA precursor of the invention in a manner such that the precursor is processed to produce mature miRNA.

As used herein, the term “mismatch” refers to one or more mismatched nucleotides in a double stranded region, unless otherwise indicated by the context.

As used herein, the term “bulge” refers to one or more unopposed nucleotides in a double stranded region, unless otherwise indicated by the context.

“Target gene” refers to the entire target gene, including exons, introns and regulatory regions such as promoters, enhancers, and terminators, 5′ and 3′ untranslated regions, the primary transcript, and the mature mRNA. “Target gene sequence” refers to either the nucleotide sequence of the sense strand of the entire target gene, including exons, introns and regulatory sequences such as promoters, enhancers, and terminators, 5′ and 3′ untranslated regions, the nucleotide sequence of the primary transcript, and/or the nucleotide sequence of the mature mRNA. The sense strand of a gene is the strand that is (partially) copied during transcription. A “target polynucleotide” refers to any genomic nucleic acid that is of interest as a target for modulation of expression.

A target gene may be a gene whose silencing has a high likelihood of resulting in a strong phenotype, preferably a knockout or null phenotype. Such target genes are often those whose protein products are involved in core cellular processes such as DNA replication, cell cycle, transcription, RNA processing, translation, protein trafficking, secretion, protein modification, protein stability and degradation, energy production, intermediary metabolism, cell structure, signal transduction, channels and transporters, and endocytosis. In a preferred embodiment, it is advantageous to select a gene for which a small decrease in expression levels results in deleterious or positive effects for the targeted organism.

“Target polynucleotide” refers to the part of a target gene which is bound or hybridized by the targeting strand (guide strand) of the at least one double-stranded silencing element (e.g., guide/passenger strand) of the interfering RNA molecule (e.g., miRNA precursor molecule). The target polynucleotide may correspond to a fragment of the whole target gene. Therefore, the target polynucleotide may comprise at least about 17, 18, 19, 20, 21, 22, 23, 24, or 25 contiguous nucleotides of the target gene. The targeting strand similarly may be at least about 17, 18, 19, 20, 21, 22, 23, 24, or 25 nucleotides long. However, the target polynucleotide and the targeting strand need not be equal in length.

The skilled person is aware of methods for identifying the most suitable target polynucleotide within the context of the full-length target gene. For example, multiple double-stranded silencing elements targeting different target polynucleotides can be synthesized and tested. Alternatively, digestion of the RNA transcript with enzymes such as RNAse H or the RNase H-like protein Argonaute can be used to determine sites of the RNA that are in a conformation susceptible to gene silencing. Target polynucleotides may also be identified using in silico approaches, for example, the use of computer algorithms designed to predict the efficacy of gene silencing based on targeting different regions within the full-length target gene.

As used herein, “operatively associated with,” “operatively linked to,” or “operably linked to,” when referring to a first nucleic acid sequence that is operatively linked to a second nucleic acid sequence, means a situation where the first nucleic acid sequence is placed in a functional relationship with the second nucleic acid sequence. For instance, a promoter is operatively linked to a coding sequence if the promoter effects the transcription or expression of the coding sequence.

A DNA “promoter” is an untranslated DNA sequence upstream of a coding region that contains the binding site for RNA polymerase and initiates transcription of the DNA. A “promoter region” can also include other elements that act as regulators of gene expression. Promoters can include, for example, constitutive, inducible, temporally regulated, developmentally regulated, chemically regulated, tissue-preferred and tissue-specific promoters for use in the preparation of recombinant nucleic acid molecules, i.e., chimeric genes. In particular aspects, a “promoter” useful with the invention is a promoter capable of initiating transcription of a nucleotide sequence in a cell of a plant.

A “chimeric gene” is a recombinant nucleic acid molecule in which a promoter or other regulatory nucleotide sequence is operatively associated with a nucleotide sequence that codes for an mRNA or which is expressed as a protein, such that the regulatory nucleotide sequence is able to regulate transcription or expression of the associated nucleotide sequence. The regulatory nucleotide sequence of the chimeric gene is not normally operatively linked to the associated nucleotide sequence as found in nature.

As used herein “sequence identity” refers to the extent to which two optimally aligned polynucleotide or peptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence.

As used herein, the phrase “substantially identical,” in the context of two nucleic acid molecules, nucleotide sequences or polypeptide sequences, refers to two or more sequences or subsequences that have at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 95%, at least about 96%, at least about 97%, at least about 98%, or at least about 99% nucleotide or amino acid residue identity, when compared and aligned for maximum correspondence, as measured using one of the following sequence comparison algorithms or by visual inspection. In some embodiments of the invention, the substantial identity exists over a region of the sequences that is at least about 50 residues/nucleotides to about 150 residues/nucleotides in length. Thus, in some embodiments of the invention, the substantial identity exists over a region of the sequences that is at least about 50, about 60, about 70, about 80, about 90, about 100, about 110, about 120, about 130, about 140, about 150, or more residues/nucleotides in length. In some particular embodiments, the sequences are substantially identical over at least about 150 residues/nucleotides. In a further embodiment, the sequences are substantially identical over the entire length of the sequences.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, Calif.). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

One example of an algorithm that is suitable for determining percent sequence identity and sequence similarity is the BLAST algorithm, which is described in Altschul et al., J. Mol. Biol. 215: 403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89: 10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleotide sequence to the reference nucleotide sequence is less than about 0.1 to less than about 0.001. Thus, in some embodiments of the invention, the smallest sum probability in a comparison of the test nucleotide sequence to the reference nucleotide sequence is less than about 0.001.

Another widely used and accepted computer program for performing sequence alignments is CLUSTALW v1.6 (Thompson, et al. Nuc. Acids Res., 22: 4673-4680, 1994). The number of matching bases or amino acids is divided by the total number of bases or amino acids, and multiplied by 100 to obtain a percent identity. For example, if two 580 base pair sequences had 145 matched bases, they would be 25 percent identical. If the two compared sequences are of different lengths, the number of matches is divided by the shorter of the two lengths. For example, if there were 100 matched amino acids between a 200 and a 400 amino acid proteins, they are 50 percent identical with respect to the shorter sequence. If the shorter sequence is less than 150 bases or 50 amino acids in length, the number of matches are divided by 150 (for nucleic acid bases) or 50 (for amino acids), and multiplied by 100 to obtain a percent identity.

In some embodiments, two nucleotide sequences can also be considered to be substantially complementary when the two sequences hybridize to each other under stringent conditions. In some representative embodiments, two nucleotide sequences considered to be substantially complementary hybridize to each other under highly stringent conditions.

The terms “stringent conditions” or “stringent hybridization conditions” include reference to conditions under which a nucleic acid will selectively hybridize to a target sequence to a detectably greater degree than other sequences (e.g., at least 2-fold over a non-target sequence), and optionally may substantially exclude binding to non-target sequences. Stringent conditions are sequence-dependent and will vary under different circumstances. By controlling the stringency of the hybridization and/or washing conditions, target sequences can be identified that can be up to 100% complementary to the reference nucleotide sequence. Alternatively, conditions of moderate or even low stringency can be used to allow some mismatching in sequences so that lower degrees of sequence similarity are detected. For example, those skilled in the art will appreciate that to function as a primer or probe, a nucleic acid sequence only needs to be sufficiently complementary to the target sequence to substantially bind thereto so as to form a stable double-stranded structure under the conditions employed. Thus, primers or probes can be used under conditions of high, moderate or even low stringency. Likewise, conditions of low or moderate stringency can be advantageous to detect homolog, ortholog and/or paralog sequences having lower degrees of sequence identity than would be identified under highly stringent conditions.

For DNA-DNA hybrids, the Tₘ can be approximated from the equation of Meinkoth and Wahl, Anal. Biochem., 138:267-84 (1984): Tₘ=81.5° C.+16.6 (log M)+0.41 (% GC)−0.61 (% formamide)−500/L; where M is the molarity of monovalent cations, % GC is the percentage of guanosine and cytosine nucleotides in the DNA, % formamide is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired degree of identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, highly stringent conditions can utilize a hybridization and/or wash at the thermal melting point (Tₘ) or 1, 2, 3 or 4° C. lower than the thermal melting point (Tₘ); moderately stringent conditions can utilize a hybridization and/or wash at 6, 7, 8, 9 or 10° C. lower than the thermal melting point (Tₘ); low stringency conditions can utilize a hybridization and/or wash at 11, 12, 13, 14, 15 or 20° C. lower than the thermal melting point (Tₘ). If the desired degree of mismatching results in a Tₘ of less than 45° C. (aqueous solution) or 32° C. (formamide solution), optionally the SSC concentration can be increased so that a higher temperature can be used. An extensive guide to the hybridization of nucleic acids is found in Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier, New York (1993); Current Protocols in Molecular Biology, chapter 2, Ausubel, et al., eds, Greene Publishing and Wiley-Interscience, New York (1995); and Green & Sambrook, In: Molecular Cloning, A Laboratory Manual, 4th Edition, Cold Spring Harbor Press, Cold Spring Harbor, N.Y. (2012).

Typically, stringent conditions are those in which the salt concentration is less than about 1.5 M Na ion, typically about 0.01 to 1.0 M Na ion concentration (or other salts) at about pH 7.0 to pH 8.3 and the temperature is at least about 30° C. for short probes (e.g., 10 to 50 nucleotides) and at least about 60° C. for longer probes (e.g., greater than 50 nucleotides). Stringent conditions may also be achieved with the addition of destabilizing agents such as formamide or Denhardt's (5 g Ficoll, 5 g polyvinylpyrrolidone, 5 g bovine serum albumin in 500 ml of water). Exemplary low stringency conditions include hybridization with a buffer solution of 30% to 35% formamide, 1 M NaCl, 1% SDS (sodium dodecyl sulfate) at 37° C. and a wash in 1× to 2×SSC (20×SSC=3.0 M NaCl/0.3 M trisodium citrate) at 50° C. to 55° C. Exemplary moderate stringency conditions include hybridization in 40% to 45% formamide, 1 M NaCl, 1% SDS at 37° C. and a wash in 0.5× to 1×SSC at 55° C. to 60° C. Exemplary high stringency conditions include hybridization in 50% formamide, 1 M NaCl, 1% SDS at 37° C. and a wash in 0.1×SSC at 60° C. to 65° C. A further non-limiting example of high stringency conditions include hybridization in 4×SSC, 5×Denhardt's, 0.1 mg/ml boiled salmon sperm DNA, and 25 mM Na phosphate at 65° C. and a wash in 0.1×SSC, 0.1% SDS at 65° C. Another illustration of high stringency hybridization conditions includes hybridization in 7% SDS, 0.5 M NaPO₄, 1 mM EDTA at 50° C. with washing in 2×SSC, 0.1% SDS at 50° C., alternatively with washing in 1×SSC, 0.1% SDS at 50° C., alternatively with washing in 0.5×SSC, 0.1% SDS at 50° C., or alternatively with washing in 0.1×SSC, 0.1% SDS at 50° C., or even with washing in 0.1×SSC, 0.1% SDS at 65° C. Those skilled in the art will appreciate that specificity is typically a function of post-hybridization washes, the relevant factors being the ionic strength and temperature of the final wash solution.

As used herein, the terms “transformation,” “transfection,” and “transduction” refer to the introduction of an exogenous/heterologous nucleic acid (RNA and/or DNA) into a host cell. A cell has been “transformed,” “transfected” or “transduced” with an exogenous/heterologous nucleic acid when such nucleic acid has been introduced or delivered into the cell.

As used herein with respect to plants and plant parts, the term “transgenic” refers to a plant, plant part or plant cell that comprises one or more exogenous nucleic acids. Generally, the exogenous nucleic acid is stably integrated within the genome such that the polynucleotide is passed on to successive generations. The exogenous nucleic acid may be integrated into the genome alone or as part of a recombinant expression cassette. “Transgenic” may be used to designate any plant, plant part or plant cell the genotype of which has been altered by the presence of an exogenous nucleic acid, including those transgenics initially so altered and those created by sexual crosses or asexual propagation from the initial transgenic. As used herein, the term “transgenic” does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition or spontaneous mutation.

The invention is directed in part to the development of synthetic miRNA precursor molecules for the delivery to plants of miRNAs that target specific nucleic acids (e.g., gene or nucleic acid targets) in a plant pest for modulating the expression of said targets in the pest, thereby providing the plant with resistance to the pest. The precursors of the invention provide a scaffold into which any guide strand can be placed for expression, e.g., a guide strand that targets a nucleic acid of interest in a plant pest.

Accordingly, one aspect of the invention relates to a synthetic plant microRNA (miRNA) precursor comprising four DCL-1 cleavage sites, the precursor comprising the following structures in loop-to-base order:

A. a terminal loop;

B. a neck stem;

C. a pair of mismatches and the flanking nucleotides surrounding a first cleavage site;

D. a pair of mismatches and/or bulges and the flanking nucleotides surrounding a second cleavage site; and/or

E. a series of mismatches and/or bulges and the flanking nucleotides between the second cleavage site and a third cleavage site;

wherein the precursor is modified at one or more of structures A, B, C, D, and E to be resistant to cleavage by a plant Dicer-like protein-1 (DCL-1) but susceptible to cleavage by Dicer or a Dicer-like protein of a plant pest.

The plant miRNA precursor of the invention may be prepared by modification of the sequence of any plant miRNA precursor, e.g., a wild-type plant miRNA precursor sequence or a consensus plant miRNA precursor sequence. For example, the starting plant miRNA precursor may be a consensus sequence developed from members of a family of miRNA precursors, e.g., a family of highly expressed precursors such as miR159.

The modification(s) at one or more of structures A, B, C, D, and E may be any number of modifications to any one or a combination of structures A, B, C, D, and E, e.g., a combination of A and B; A and C; A and D; A and E; B and C; B and D; B and E; C and D; C and E; A, B, and C; A, B, and D; A, B, and E; A, C, and D; A, C, and E; A, D, and E; B, C, and E; B, C, and E; C, D, and E; A, B, C, and D; A, B, C, and E; A, B, D, and E; B, C, D, and E; or A, B, C, D, and E. The modification(s) may be a change in sequence, length, symmetry (i.e., mismatches and/or bulges), or a combination thereof. The changes may be made by insertion, deletion, and/or substitution of one or more nucleotides in the precursor sequence. In some embodiments, the number of nucleotides that are inserted, deleted, and/or substituted may be 1, 2, 3, 4, 5, 6, 7, 8, 9, 10, 11, 12, 13, 14, 15, 16, 17, 18, 19, or 20 or more. In other embodiments, the number of nucleotides that are inserted, deleted, and/or substituted may be 50 or less, 40 or less, 30, or less, 20 or less, 15 or less, or 10 or less.

In some embodiments, the modification comprises a modification of structure A, e.g., a change in the length and/or sequence of the terminal loop. In some embodiments, the terminal loop is modified to a length of about 10 to about 50 nucleotides. In certain embodiments, the length of the terminal loop is shortened relative to the length of the terminal loop in the starting precursor sequence, e.g., by 1, 2, 3, 4, 5, 6, 7, 8, 9, or 10 or more nucleotides relative to the starting precursor sequence. In other embodiments, the length of the terminal loop is lengthened relative to the length of the terminal loop in the starting precursor sequence, e.g., by 1, 2, 3, 4, 5, 6, 7, 8, 9, or 10 or more nucleotides relative to the starting precursor sequence. In some embodiments, the terminal loop is modified to be 10-20, 20-30, 30-40, 40-50, 10-30, 20-40, or 30-50 nucleotides in length. In some embodiments, the terminal loop is modified to add one or more additional loops or other secondary structures. In certain embodiments, the length of the terminal loop is modified to decrease the thermal stability of miRNA precursor, e.g., to have a folding energy (ΔG) larger than −100 kcal/mol.

In some embodiments, the modification comprises a modification of structure B, e.g., a change in the length and/or sequence of the neck stem. In some embodiments, the terminal loop is modified to a length of about 2 to about 10 base pairs. In certain embodiments, the length of the neck stem is shortened relative to the length of the neck stem in the starting precursor sequence, e.g., by 1, 2, 3, 4, 5, 6, 7, 8, 9, or 10 or more base pairs relative to the starting precursor sequence. In other embodiments, the length of the neck stem is lengthened relative to the length of the neck stem in the starting precursor sequence, e.g., by 1, 2, 3, 4, 5, 6, 7, 8, 9, or 10 or more base pairs relative to the starting precursor sequence. In some embodiments, the neck stem is modified to be 2-5, 3-7, or 6-10 base pairs in length. In certain embodiments, the length of the neck stem is modified to decrease the stability of miRNA precursor, e.g., to have a folding energy (ΔG) larger than −100 kcal/mol.

In some embodiments, the modification comprises a modification of structure C, e.g., a change in the sequence, length, symmetry, or a combination thereof in one or both mismatches of the pair of mismatches and the flanking nucleotides surrounding the first cleavage site. The term “flanking nucleotides,” as used herein with respect to structure C, refers to the 2-3 nucleotides 5′ of the pair of mismatches, 2-3 nucleotides 3′ of the pair of mismatches, and the nucleotides between the two mismatches. The modification can be to one or both of the mismatches and/or the nucleotides between the two mismatches. The present invention encompasses embodiments in which the cleavage site is functional (i.e., is cleaved by DCL-1) or nonfunctional (i.e., is not cleaved by DCL-1), e.g., due to the modification of the mismatches. In some embodiments, the modification comprises changing the nucleotide sequence of one or both mismatches. In some embodiments, the modification comprises decreasing or increasing the length of one or both mismatches, e.g., by 1, 2, 3, or 4 nucleotides. In some embodiments, the modification comprises deleting one or more nucleotides in one or both mismatches to create one or more bulges, e.g., by deleting or inserting one or more nucleotides on one strand but not the other strand. In certain embodiments, the mismatch closest to the terminal loop is converted to a bulge. In some embodiments, the modification comprises decreasing or increasing the number of basepairs between the two mismatches. The modifications may involve eliminating or altering G-C basepairs, e.g., in the nucleotides between the two mismatches, in order to remove strong basepairs and destabilize the structure. In some embodiments, the modifications may include changing a GUUU sequence in a mismatch to any triple nucleotide sequence.

In some embodiments, the modification comprises a modification of structure D, e.g., a change in the sequence, length, symmetry, or a combination thereof in one or both mismatches and/or bulges and the flanking nucleotides of the pair of mismatches and/or bulges surrounding the second cleavage site. The term “flanking nucleotides,” as used herein with respect to structure D, refers to the 2-3 nucleotides 5′ of the pair of mismatches and/or bulges, 2-3 nucleotides 3′ of the pair of mismatches and/or bulges, and the nucleotides between the two mismatches and/or bulges. In some embodiments, the modification comprises changing the nucleotide sequence of one or both mismatches and/or bulges. In some embodiments, the modification comprises eliminating one or both of the mismatches and/or bulges. In some embodiments, the modification comprises converting a nucleotide base pair between the two mismatches and/or bulges to a mismatch or removing the nucleotide basepair between the two mismatches. In some embodiments, the modification comprises deleting one or more nucleotides to convert one or more mismatches into a bulge. In certain embodiments, a U-rich region is modified to reduce the number of uridines in the cleavage site area. The modifications may involve eliminating or altering G-C basepairs, e.g., in the nucleotides between the two mismatches, in order to remove strong basepairs and destabilize the structure.

In some embodiments, the modification comprises a modification of structure E, e.g., a change in the change in the sequence, length, symmetry, or a combination thereof in the series of mismatches and/or bulges and the flanking nucleotides between the second cleavage site and a third cleavage site. The term “flanking nucleotides,” as used herein with respect to structure E, refers to the 2-3 nucleotides 5′ and 3′ of each mismatch and bulge. In some embodiments, structure E contains 2-4 mismatches and/or bulges, e.g., 3 mismatches and/or bulges. In some embodiments, the modification comprises changing the nucleotide sequence of one or more of the mismatches and/or bulges. In some embodiments, the modification comprises changing the length of one or more of the mismatches and/or bulges. In some embodiments, the modification comprises eliminating one or more of the mismatches and/or bulges. In some embodiments, the modification comprises converting a mismatch to a bulge. In some embodiments, the modification comprises converting a bulge to a mismatch.

In certain embodiments, the modifications to the miRNA precursor starting sequence are designed to make the precursor more unstable, e.g., to increase the folding energy (ΔG) required for the precursor to form the hairpin structure. In some embodiments, the synthetic plant miRNA precursor has a folding energy (ΔG) larger than −100 kcal/mol, e.g., larger than −90, −80, or −70 kcal/mol. The increased instability may be due to a single modification in one of structures A, B, C, D, and E or a combination of modifications in one or more of structures A, B, C, D, and E.

The synthetic DCL-1 resistant miRNA precursor scaffold of the present invention is advantageously used to target plant pests that might feed on or otherwise damage the plant. Thus, in one aspect of the invention, the synthetic plant miRNA precursor further comprises a miRNA guide strand and passenger strand targeted to a target polynucleotide or target gene of a plant pest. In some embodiments, the miRNA guide strand is about 40% to about 100% complementary to a target polynucleotide or target gene of a plant pest, e.g., at least about 40%, 50%, 60%, 70%, 80%, 85%, 90%, 95%, 96%, 97%, 98%, 99% or more complementary.

The plant pest may be any pest that is known to damage a plant and that is capable of taking up a miRNA precursor that is present in or on the plant. Pests include, without limitation, insects, nematodes, mites, ticks, gastropods, fungi, and bacteria.

In some embodiments, the pest is an insect. Insect pests include without limitation insects selected from the orders Coleoptera, Diptera, Hymenoptera, Lepidoptera, Mallophaga, Homoptera, Hemiptera, Orthroptera, Thysanoptera, Dermaptera, Isoptera, Anoplura, Siphonaptera, Trichoptera, and the like. In some embodiments, insect pests include without limitation Ostrinia nubilalis (European corn borer), Plutella xylostella (diamondback moth), Spodoptera frugiperda (fall armyworm), Agrotis ipsilon (black cutworm), Agrotis orthogonia (pale western cutworm), Striacosta albicosta (western bean cutworm), Helicoverpa zea (corn earworm), Heliothis virescens (tobacco budworm), Spodoptera exigua (beet armyworm), Helicoverpa punctigera (native budworm), Helicoverpa armigera (cotton bollworm), Manduca sexta (tobacco hornworm), Trichoplusia ni (cabbage looper), Pectinophora gossypiella (pink bollworm), Diatraea grandiosella (southwestern corn borer), Diatraea saccharalis (sugarcane borer), Elasmopalpus lignosellus (lesser cornstalk borer), Psuedoplusia includens (soybean looper), Anticarsia gemmatalis (velvetbean caterpillar), Plathypena scabra (green cloverworm), Homoeosoma electellum (sunflower head moth), Cochylis hospes (banded sunflower moth), Diabrotica virgifera virgifera (western corn rootworm), or any combination thereof.

In some embodiments, the pest is a nematode. The term “nematode” as used herein encompasses any organism that is now known or later identified that is classified in the animal kingdom, phylum Nematoda, including without limitation nematodes within class Adenophorea (including for example, orders Enoplida, Isolaimida, Mononchida, Dorylaimida, Trichocephalida, Mermithida, Muspiceida, Araeolaimida, Chromadorida, Desmoscolecida, Desmodorida and Monhysterida) and/or class Secernentea (including, for example, orders Rhabdita, Strongylida, Ascaridida, Spirurida, Camallanida, Diplogasterida, Tylenchida and Aphelenchida).

Nematodes include but are not limited to parasitic nematodes such as root-knot nematodes, cyst nematodes and/or lesion nematodes.

Exemplary genera of nematodes according to the present invention include but are not limited to, Meloidogyne (root-knot nematodes), Heterodera (cyst nematodes), Globodera (cyst nematodes), Radopholus (burrowing nematodes), Rotylenchulus (reniform nematodes), Pratylenchus (lesion nematodes), Aphelenchoides (foliar nematodes), Helicotylenchus (spiral nematodes), Hoplolaimus (lance nematodes), Paratrichodorus (stubby-root nematodes), Longidorus, Nacobbus (false root-knot nematodes), Subanguina, Belonlaimus (sting nematodes), Criconemella, Criconemoides (ring nematodes), Ditylenchus, Dolichodorus, Hemicriconemoides, Hemicycliophora, Hirschmaniella, Hypsoperine, Macroposthonia, Melinius, Punctodera, Quinisulcius, Scutellonema, Xiphinema (dagger nematodes), Tylenchorhynchus (stunt nematodes), Tylenchulus, Bursaphelenchus (round worms), and any combination thereof.

Exemplary plant parasitic nematodes according to the present invention include, but are not limited to, Belonolaimus gracilis, Belonolaimus longicaudatus, Bursaphelenchus xylophilus (pine wood nematode), Criconemoides ornata, Ditylenchus destructor (potato rot nematode), Ditylenchus dipsaci (stem and bulb nematode), Globodera pallida (potato cyst nematode), Globodera rostochiensis (golden nematode), Heterodera glycines (soybean cyst nematode), Heterodera schachtii (sugar beet cyst nematode); Heterodera zeae (corn cyst nematode), Heterodera avenae (cereal cyst nematode), Heterodera carotae, Heterodera trifolii, Hoplolaimus columbus, Hoplolaimus galeatus, Hoplolaimus magnistylus, Longidorus breviannulatus, Meloidogyne arenaria, Meloidogyne chitwoodi, Meloidogyne hapla, Meloidogyne incognita, Meloidogyne javanica, Mesocriconema xenoplax, Nacobbus aberrans, Naccobus dorsalis, Paratrichodorus christiei, Paratrichodorus minor, Pratylenchus brachyurus, Pratylenchus crenatus, Pratylenchus hexincisus, Pratylenchus neglectus, Pratylenchus penetrans, Pratylenchus projectus, Pratylenchus scribneri, Pratylenchus tenuicaudatus, Pratylenchus thornei, Pratylenchus zeae, Punctodera chaccoensis, Quinisulcius acutus, Radopholus similis, Rotylenchulus reniformis, Tylenchorhynchus dubius, Tylenchulus semipenetrans (citrus nematode), Siphinema americanum, X. Mediterraneum, and any combination of the foregoing.

The target polynucleotide or target gene of a plant pest may be any polynucleotide or gene for which modulation of expression will adversely affect (reduce) the damage induced by the pest. Modulation of expression (e.g., gene silencing) may result in one or more of (but not limited to) the following attributes: reduction in feeding by the pest, reduction in viability of the pest, death of the pest, inhibition of differentiation and development of the pest, absence of or reduced capacity for sexual reproduction by the pest, muscle formation, juvenile hormone formation, juvenile hormone regulation, ion regulation and transport, maintenance of cell membrane potential, amino acid biosynthesis, amino acid degradation, sperm formation, pheromone synthesis, pheromone sensing, antennae formation, wing formation, leg formation, development and differentiation, egg formation, larval maturation, digestive enzyme formation, haemolymph synthesis, haemolymph maintenance, neurotransmission, cell division, energy metabolism, respiration, apoptosis, and any component of a eukaryotic cells' cytoskeletal structure, such as, for example, actins and tubulins. Any one or any combination of these attributes can result in effective inhibition of pest infestation, and in the case of a plant pest, inhibition of plant infestation.

The cell comprising the target polynucleotide or gene may be derived from or contained in any organism. The organism may be a plant, animal, protozoan, bacterium, virus, or fungus. The plant may be a monocot, dicot or gymnosperm; the animal may be a vertebrate or invertebrate. Preferred microbes are those used in agriculture or by industry, and those that are pathogenic for plants or animals. Fungi include organisms in both the mold and yeast morphologies.

Suitable target polynucleotides and genes in plant pests are well known in the art. Examples include, without limitation, histone genes, Inhibitor of Apoptosis Protein genes, ribosomal protein genes, glutamate tRNA synthetase genes, and genes that remodel the structure of chromatin.

A synthetic precursor molecule of the invention does not comprise a 100% identity to any wild type miRNA precursor molecule (e.g., does not comprise a 100% identity to MIR159, MIR156, MIR319, and the like). In some aspects, a synthetic precursor molecule of the invention does not comprise a 100% identity to 50, 100, 150, 200 or 250 contiguous nucleotides of any wild type miRNA precursor molecule.

The present invention provides a miRNA precursor into which any guide strand can be placed for high efficiency expression in plants. Thus, the present invention provides synthetic precursor molecules comprising target-specific amiRNAs or miRNA guide strands that can be used in modulating the expression of a target gene or target polynucleotide in a plant pest. In some aspects, a miRNA guide strand of a synthetic DCL-1 resistant miRNA precursor molecule of the invention can be about 60% to about 100% (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% or any value or range therein) complementary to a target gene or target polynucleotide (or fragment thereof) in a plant pest. A miRNA guide strand of the invention forms a double stranded (ds) RNA molecule through complementary base pairing with a miRNA passenger strand. In some embodiments, the miRNA passenger strand is designed to base pair with the miRNA guide strand such that the dsRNA formed comprises, consists essentially of, or consists of three single nucleotide mismatches with the first mismatch formed between the 5′ most nucleotide U of the guide strand and the 3′ most nucleotide of the passenger strand, the second single nucleotide mismatch formed six nucleotides (including the mismatched nucleotide) upstream (5′) of the first mismatch and a third single nucleotide mismatch formed four nucleotides (including the mismatched nucleotide) upstream (5′) of the second mismatch. Accordingly, in some aspects, a miRNA passenger strand and a miRNA guide strand of the synthetic DCL-1 resistant miRNA precursor molecule of the invention have about 80 to 90% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, or any value or range therein) complementarity to one another.

In some aspects, the length of a amiRNA (guide strand) can be about 17 to about 25 nucleotides in length (e.g., 17, 18, 19, 20, 21, 22, 23, 24, or 25 nucleotides in length, and/or any range therein).

In some aspects, a recombinant nucleic acid molecule comprises a nucleotide sequence encoding a synthetic DCL-1 resistant miRNA precursor of the invention. In some aspects, the invention provides an expression cassette or vector comprising a nucleotide sequence encoding a synthetic DCL-1 resistant miRNA precursor of the invention. In some aspects of the invention, the nucleotide sequence encoding a synthetic DCL-1 resistant miRNA precursor of the invention can be RNA or DNA.

In some aspects, the nucleotide sequences and/or recombinant nucleic acid molecules of the invention can be operatively linked to one or more promoter sequences for expression in host cells (e.g., plant cells). Promoters useful with the invention include, but are not limited to, those that drive expression of a nucleotide sequence constitutively, those that drive expression when induced, and those that drive expression in a tissue- or developmentally-specific manner. These various types of promoters are known in the art.

The choice of promoter will vary depending on the temporal and spatial requirements for expression, and also depending on the host cell to be transformed. Thus, for example, expression of the nucleotide sequences of the invention can be in any plant and/or plant part, (e.g., in cells, in leaves, in stalks or stems, in ears, in inflorescences (e.g., spikes, panicles, cobs, etc.), in roots, seeds and/or seedlings, and the like). In many cases, however, protection against more than one type of pest is sought, and thus expression in multiple tissues is desirable. Although many promoters from dicotyledons have been shown to be operational in monocotyledons and vice versa, ideally dicotyledonous promoters are selected for expression in dicotyledons, and monocotyledonous promoters for expression in monocotyledons. However, there is no restriction to the provenance of selected promoters; it is sufficient that they are operational in driving the expression of the nucleotide sequences in the desired cell.

Examples of constitutive promoters include, but are not limited to, cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter. The constitutive promoter derived from ubiquitin accumulates in many cell types. Ubiquitin promoters have been cloned from several plant species for use in transgenic plants, for example, sunflower (Binet et al., 1991. Plant Science 79: 87-94), maize (Christensen et al., 1989. Plant Molec. Biol. 12: 619-632), and arabidopsis (Norris et al., 1993. Plant Molec. Biol. 21:895-906). The maize ubiquitin promoter (UbiP) has been developed in transgenic monocot systems and its sequence and vectors constructed for monocot transformation are disclosed in the patent publication EP 0 342 926. The ubiquitin promoter is suitable for the expression of the nucleotide sequences of the invention in transgenic plants, especially monocotyledons. Further, the promoter expression cassettes described by McElroy et al. (Mol. Gen. Genet. 231: 150-160 (1991)) can be easily modified for the expression of the nucleotide sequences of the invention and are particularly suitable for use in monocotyledonous hosts.

In some embodiments, tissue specific/tissue preferred promoters can be used. Tissue specific or preferred expression patterns include, but are not limited to, green tissue specific or preferred, root specific or preferred, stem specific or preferred, and flower specific or preferred. Promoters suitable for expression in green tissue include many that regulate genes involved in photosynthesis and many of these have been cloned from both monocotyledons and dicotyledons. In one embodiment, a promoter useful with the invention is the maize PEPC promoter from the phosphoenol carboxylase gene (Hudspeth & Grula, Plant Molec. Biol. 12:579-589 (1989)). Non-limiting examples of tissue-specific promoters include those associated with genes encoding the seed storage proteins (such as β-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP Patent No. 255378). Tissue-specific or tissue-preferential promoters useful for the expression of the nucleotide sequences of the invention in plants, particularly maize, include but are not limited to those that direct expression in root, pith, leaf or pollen. Such promoters are disclosed, for example, in WO 93/07278, herein incorporated by reference in its entirety. Other non-limiting examples of tissue specific or tissue preferred promoters useful with the invention the cotton rubisco promoter disclosed in U.S. Pat. No. 6,040,504; the rice sucrose synthase promoter disclosed in U.S. Pat. No. 5,604,121; the root specific promoter described by de Framond (FEBS 290:103-106 (1991); EP 0 452 269 to Ciba-Geigy); the stem specific promoter described in U.S. Pat. No. 5,625,136 (to Ciba-Geigy) and which drives expression of the maize trpA gene; and the cestrum yellow leaf curling virus promoter disclosed in WO 01/73087, all incorporated by reference.

Additional examples of tissue-specific/tissue preferred promoters include, but are not limited to, the root-specific promoters RCc3 (Jeong et al., Plant Physiol. 153:185-197 (2010)) and RB7 (U.S. Pat. No. 5,459,252), the lectin promoter (Lindstrom et al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAMS) (Vander Mijnsbrugge et al. (1996) Plant and Cell Physiology, 37(8):1108-1115), corn light harvesting complex promoter (Bansal et al. (1992) Proc. Natl. Acad. Sci. USA 89:3654-3658), corn heat shock protein promoter (O'Dell et al. (1985) EMBO J. 5:451-458; and Rochester et al. (1986) EMBO J. 5:451-458), pea small subunit RuBP carboxylase promoter (Cashmore, “Nuclear genes encoding the small subunit of ribulose-1,5-bisphosphate carboxylase” pp. 29-39 In: Genetic Engineering of Plants (Hollaender ed., Plenum Press 1983; and Poulsen et al. (1986) Mol. Gen. Genet. 205:193-200), Ti plasmid mannopine synthase promoter (Langridge et al. (1989) Proc. Natl. Acad. Sci. USA 86:3219-3223), Ti plasmid nopaline synthase promoter (Langridge et al. (1989), supra), petunia chalcone isomerase promoter (van Tunen et al. (1988) EMBO J. 7:1257-1263), bean glycine rich protein 1 promoter (Keller et al. (1989) Genes Dev. 3:1639-1646), truncated CaMV 35S promoter (O'Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al. (1987) Mol. Gen. Genet. 207:90-98; Langridge et al. (1983) Cell 34:1015-1022; Reina et al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), α-tubulin cab promoter (Sullivan et al. (1989) Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J. 10:2605-2612). In some particular embodiments, the nucleotide sequences of the invention are operatively associated with a root-preferred promoter.

Particularly useful for seed-specific expression is the pea vicilin promoter (Czako et al. (1992) Mol. Gen. Genet. 235:33-40; as well as the seed-specific promoters disclosed in U.S. Pat. No. 5,625,136. Useful promoters for expression in mature leaves are those that are switched on at the onset of senescence, such as the SAG promoter from Arabidopsis (Gan et al. (1995) Science 270:1986-1988).

In addition, promoters functional in plastids can be used. Non-limiting examples of such promoters include the bacteriophage T3 gene 9 5′ UTR and other promoters disclosed in U.S. Pat. No. 7,579,516. Other promoters useful with the invention include but are not limited to the S-E9 small subunit RuBP carboxylase promoter and the Kunitz trypsin inhibitor gene promoter (Kti3).

In some embodiments of the invention, inducible promoters can be used. Thus, for example, chemical-regulated promoters can be used to modulate the expression of a gene in a plant through the application of an exogenous chemical regulator. Regulation of the expression of nucleotide sequences of the invention via promoters that are chemically regulated enables the polypeptides of the invention to be synthesized only when the crop plants are treated with the inducing chemicals. Depending upon the objective, the promoter may be a chemical-inducible promoter, where application of a chemical induces gene expression, or a chemical-repressible promoter, where application of the chemical represses gene expression.

Chemical inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1 a promoter, which is activated by salicylic acid (e.g., the PR1a system), steroid-responsive promoters (see, e.g., the glucocorticoid-inducible promoter in Schena et al. (1991) Proc. Natl. Acad. Sci. USA 88, 10421-10425 and McNellis et al. (1998) Plant J. 14, 247-257) and tetracycline-inducible and tetracycline-repressible promoters (see, e.g., Gatz et al. (1991) Mol. Gen. Genet. 227, 229-237, and U.S. Pat. Nos. 5,814,618 and 5,789,156, Lac repressor system promoters, copper-inducible system promoters, salicylate-inducible system promoters (e.g., the PR1a system), glucocorticoid-inducible promoters (Aoyama et al. (1997) Plant J. 11:605-612), and ecdysone-inducible system promoters.

Other non-limiting examples of inducible promoters include ABA- and turgor-inducible promoters, the auxin-binding protein gene promoter (Schwob et al. (1993) Plant J. 4:423-432), the UDP glucose flavonoid glycosyl-transferase promoter (Ralston et al. (1988) Genetics 119:185-197), the MPI proteinase inhibitor promoter (Cordero et al. (1994) Plant J. 6:141-150), and the glyceraldehyde-3-phosphate dehydrogenase promoter (Kohler et al. (1995) Plant Mol. Biol. 29:1293-1298; Martinez et al. (1989) J. Mol. Biol. 208:551-565; and Quigley et al. (1989) J. Mol. Evol. 29:412-421). Also included are the benzene sulphonamide-inducible (U.S. Pat. No. 5,364,780) and alcohol-inducible (Int'l Patent Application Publication Nos. WO 97/06269 and WO 97/06268) systems and glutathione S-transferase promoters. Likewise, one can use any of the inducible promoters described in Gatz (1996) Current Opinion Biotechnol. 7:168-172 and Gatz (1997) Annu. Rev. Plant Physiol. Plant Mol. Biol. 48:89-108. Other chemically inducible promoters useful for directing the expression of the nucleotide sequences of this invention in plants are disclosed in U.S. Pat. No. 5,614,395 herein incorporated by reference in its entirety. Chemical induction of gene expression is also detailed in the published application EP 0 332 104 (to Ciba-Geigy) and U.S. Pat. No. 5,614,395. In some embodiments, a promoter for chemical induction can be the tobacco PR-1a promoter.

In further aspects, the nucleotide sequences of the invention can be operatively associated with a promoter that is wound inducible or inducible by pest infection (e.g., a nematode plant pest). Numerous promoters have been described which are expressed at wound sites and/or at the sites of pest attack (e.g., insect/nematode feeding) or phytopathogen infection. Ideally, such a promoter should be active only locally at or adjacent to the sites of attack, and in this way expression of the nucleotide sequences of the invention will be focused in the cells that are being invaded. Such promoters include, but are not limited to, those described by Stanford et al., Mol. Gen. Genet. 215:200-208 (1989), Xu et al. Plant Molec. Biol. 22:573-588 (1993), Logemann et al. Plant Cell 1:151-158 (1989), Rohrmeier and Lehle, Plant Molec. Biol. 22:783-792 (1993), Firek et al. Plant Molec. Biol. 22:129-142 (1993), Warner et al. Plant J. 3:191-201 (1993), U.S. Pat. No. 5,750,386, U.S. Pat. No. 5,955,646, U.S. Pat. No. 6,262,344, U.S. Pat. No. 6,395,963, U.S. Pat. No. 6,703,541, U.S. Pat. No. 7,078,589, U.S. Pat. No. 7,196,247, U.S. Pat. No. 7,223,901, and U.S. Patent Application Publication 2010043102.

As used herein, “expression cassette” means a nucleic acid molecule comprising a nucleotide sequence of interest (e.g., the nucleotide sequences encoding the synthetic DCL-1 resistant miRNA precursor molecules of the invention), wherein said nucleotide sequence is operatively associated with at least a control sequence (e.g., a promoter). Thus, some embodiments of the invention provide expression cassettes designed to express the nucleotides sequences of the invention (e.g., the nucleotide sequences encoding the synthetic DCL-1 resistant miRNA precursor molecules of the invention). In this manner, for example, one or more plant promoters operatively associated with one or more nucleotide sequences of the invention are provided in expression cassettes for expression in an organism or cell thereof (e.g., a plant, plant part and/or plant cell).

An expression cassette comprising a nucleotide sequence of interest may be chimeric, meaning that at least one of its components is heterologous with respect to at least one of its other components. An expression cassette may also be one that is naturally occurring but has been obtained in a recombinant form useful for heterologous expression. Typically, however, the expression cassette is heterologous with respect to the host, i.e., the particular nucleic acid sequence of the expression cassette does not occur naturally in the host cell and must have been introduced into the host cell or an ancestor of the host cell by a transformation event.

In addition to the promoters operatively linked to the nucleotide sequences of the invention, an expression cassette of the invention can also include other regulatory sequences. As used herein, “regulatory sequences” means nucleotide sequences located upstream (5′ non-coding sequences), within or downstream (3′ non-coding sequences) of a coding sequence, and which influence the transcription, RNA processing or stability, or translation of the associated coding sequence. Regulatory sequences include, but are not limited to, promoters, enhancers, introns, translation leader sequences, termination signals, and polyadenylation signal sequences.

For purposes of the invention, the regulatory sequences or regions can be native/analogous to the plant, plant part and/or plant cell and/or the regulatory sequences can be native/analogous to the other regulatory sequences. Alternatively, the regulatory sequences may be heterologous to the plant (and/or plant part and/or plant cell) and/or to each other (i.e., the regulatory sequences). Thus, for example, a promoter can be heterologous when it is operatively linked to a polynucleotide from a species different from the species from which the polynucleotide was derived. Alternatively, a promoter can also be heterologous to a selected nucleotide sequence if the promoter is from the same/analogous species from which the polynucleotide is derived, but one or both (i.e., promoter and/or polynucleotide) are substantially modified from their original form and/or genomic locus, and/or the promoter is not the native promoter for the operably linked polynucleotide.

A number of non-translated leader sequences derived from viruses are known to enhance gene expression. Specifically, leader sequences from Tobacco Mosaic Virus (TMV, the “ω-sequence”), Maize Chlorotic Mottle Virus (MCMV) and Alfalfa Mosaic Virus (AMV) have been shown to be effective in enhancing expression (Gallie et al. (1987) Nucleic Acids Res. 15:8693-8711; and Skuzeski et al. (1990) Plant Mol. Biol. 15:65-79). Other leader sequences known in the art include, but are not limited to, picornavirus leaders such as an encephalomyocarditis (EMCV) 5′ noncoding region leader (Elroy-Stein et al. (1989) Proc. Natl. Acad. Sci. USA 86:6126-6130); potyvirus leaders such as a Tobacco Etch Virus (TEV) leader (Allison et al. (1986) Virology 154:9-20); Maize Dwarf Mosaic Virus (MDMV) leader (Allison et al. (1986), supra); human immunoglobulin heavy-chain binding protein (BiP) leader (Macejak & Samow (1991) Nature 353:90-94); untranslated leader from the coat protein mRNA of AMV (AMV RNA 4; Jobling & Gehrke (1987) Nature 325:622-625); tobacco mosaic TMV leader (Gallie et al. (1989) Molecular Biology of RNA 237-256); and MCMV leader (Lommel et al. (1991) Virology 81:382-385). See also, Della-Cioppa et al. (1987) Plant Physiol. 84:965-968.

An expression cassette also can optionally include a transcriptional and/or translational termination region (i.e., termination region) that is functional in plants. A variety of transcriptional terminators are available for use in expression cassettes and are responsible for the termination of transcription beyond the heterologous nucleotide sequence of interest and correct mRNA polyadenylation. The termination region may be native to the transcriptional initiation region, may be native to the operably linked nucleotide sequence of interest, may be native to the plant host, or may be derived from another source (i.e., foreign or heterologous to the promoter, the nucleotide sequence of interest, the plant host, or any combination thereof). Appropriate transcriptional terminators include, but are not limited to, the CAMV 35S terminator, the tml terminator, the nopaline synthase terminator and/or the pea rbcs E9 terminator. These can be used in both monocotyledons and dicotyledons. In addition, a coding sequence's native transcription terminator can be used.

An expression cassette of the invention also can include a nucleotide sequence for a selectable marker, which can be used to select a transformed plant, plant part and/or plant cell. As used herein, “selectable marker” means a nucleotide sequence that when expressed imparts a distinct phenotype to the plant, plant part and/or plant cell expressing the marker and thus allows such transformed plants, plant parts and/or plant cells to be distinguished from those that do not have the marker. Such a nucleotide sequence may encode either a selectable or screenable marker, depending on whether the marker confers a trait that can be selected for by chemical means, such as by using a selective agent (e.g., an antibiotic, herbicide, or the like), or on whether the marker is simply a trait that one can identify through observation or testing, such as by screening (e.g., the R-locus trait). Of course, many examples of suitable selectable markers are known in the art and can be used in the expression cassettes described herein.

Examples of selectable markers include, but are not limited to, a nucleotide sequence encoding neo or nptII, which confers resistance to kanamycin, G418, and the like (Potrykus et al. (1985) Mol. Gen. Genet. 199:183-188); a nucleotide sequence encoding bar, which confers resistance to phosphinothricin; a nucleotide sequence encoding an altered 5-enolpyruvylshikimate-3-phosphate (EPSP) synthase, which confers resistance to glyphosate (Hinchee et al. (1988) Biotech. 6:915-922); a nucleotide sequence encoding a nitrilase such as bxn from Klebsiella ozaenae that confers resistance to bromoxynil (Stalker et al. (1988) Science 242:419-423); a nucleotide sequence encoding an altered acetolactate synthase (ALS) that confers resistance to imidazolinone, sulfonylurea or other ALS-inhibiting chemicals (EP Patent Application No. 154204); a nucleotide sequence encoding a methotrexate-resistant dihydrofolate reductase (DHFR) (Thillet et al. (1988) J. Biol. Chem. 263:12500-12508); a nucleotide sequence encoding a dalapon dehalogenase that confers resistance to dalapon; a nucleotide sequence encoding a mannose-6-phosphate isomerase (also referred to as phosphomannose isomerase (PMI)) that confers an ability to metabolize mannose (U.S. Pat. Nos. 5,767,378 and 5,994,629); a nucleotide sequence encoding an altered anthranilate synthase that confers resistance to 5-methyl tryptophan; and/or a nucleotide sequence encoding hph that confers resistance to hygromycin. One of skill in the art is capable of choosing a suitable selectable marker for use in an expression cassette of the invention.

Additional selectable markers include, but are not limited to, a nucleotide sequence encoding β-glucuronidase or uidA (GUS) that encodes an enzyme for which various chromogenic substrates are known; an R-locus nucleotide sequence that encodes a product that regulates the production of anthocyanin pigments (red color) in plant tissues (Dellaporta et al., “Molecular cloning of the maize R-nj allele by transposon-tagging with Ac,” pp. 263-282 In: Chromosome Structure and Function: Impact of New Concepts, 18th Stadler Genetics Symposium (Gustafson & Appels eds., Plenum Press 1988)); a nucleotide sequence encoding β-lactamase, an enzyme for which various chromogenic substrates are known (e.g., PADAC, a chromogenic cephalosporin) (Sutcliffe (1978) Proc. Natl. Acad. Sci. USA 75:3737-3741); a nucleotide sequence encoding xylE that encodes a catechol dioxygenase (Zukowsky et al. (1983) Proc. Natl. Acad. Sci. USA 80:1101-1105); a nucleotide sequence encoding tyrosinase, an enzyme capable of oxidizing tyrosine to DOPA and dopaquinone, which in turn condenses to form melanin (Katz et al. (1983) J. Gen. Microbiol. 129:2703-2714); a nucleotide sequence encoding β-galactosidase, an enzyme for which there are chromogenic substrates; a nucleotide sequence encoding luciferase (lux) that allows for bioluminescence detection (Ow et al. (1986) Science 234:856-859); a nucleotide sequence encoding aequorin, which may be employed in calcium-sensitive bioluminescence detection (Prasher et al. (1985) Biochem. Biophys. Res. Comm. 126:1259-1268); or a nucleotide sequence encoding green fluorescent protein (Niedz et al. (1995) Plant Cell Reports 14:403-406). One of skill in the art is capable of choosing a suitable selectable marker for use in an expression cassette of the invention.

An expression cassette of the invention also can include nucleotide sequences that encode other desired traits. Such desired traits can be other nucleotide sequences which confer nematode resistance, insect resistance, disease resistance, or which confer other agriculturally desirable traits. Such nucleotide sequences can be stacked with any combination of nucleotide sequences to create plants, plant parts or plant cells having the desired phenotype. Stacked combinations can be created by any method including, but not limited to, cross breeding plants by any conventional methodology, or by genetic transformation. If stacked by genetically transforming the plants, nucleotide sequences encoding additional desired traits can be combined at any time and in any order. For example, a transgenic plant comprising one or more desired traits can be used as the target to introduce further traits by subsequent transformation. The additional nucleotide sequences can be introduced simultaneously in a co-transformation protocol with a nucleotide sequence, nucleic acid molecule, nucleic acid construct, and/or composition of the invention, provided by any combination of expression cassettes. For example, if two nucleotide sequences will be introduced, they can be incorporated in separate cassettes (trans) or can be incorporated on the same cassette (cis). Expression of the nucleotide sequences can be driven by the same promoter or by different promoters. It is further recognized that nucleotide sequences can be stacked at a desired genomic location using a site-specific recombination system. See, e.g., Int'l Patent Application Publication Nos. WO 99/25821; WO 99/25854; WO 99/25840; WO 99/25855 and WO 99/25853. In representative embodiments, a nucleic acid molecule, expression cassette or vector of the invention can comprise a transgene that confers resistance to one or more herbicides, optionally glyphosate-, sulfonylurea-, imidazolinione-, dicamba-, glufisinate-, phenoxy proprionic acid-, cycloshexome-, traizine-, benzonitrile-, and/or broxynil-resistance; a transgene that confers resistance to one or more pests, optionally bacterial-, fungal-, gastropod-, insect-, nematode-, oomycete-, phytoplasma-, protozoa-, and/or viral-resistance, and/or a transgene that confers resistance to one or more diseases.

In addition to expression cassettes, the nucleic acid molecules and nucleotide sequences described herein can be used in connection with vectors. The term “vector” refers to a composition for transferring, delivering or introducing a nucleic acid (or nucleic acids) into a cell. A vector comprises a nucleic acid molecule comprising the nucleotide sequence(s) to be transferred, delivered or introduced. Vectors for use in transformation of plants and other organisms are well known in the art. Non-limiting examples of general classes of vectors include a viral vector including but not limited to an adenovirus vector, a retroviral vector, an adeno-associated viral vector, a plasmid vector, a phage vector, a phagemid vector, a cosmid, a fosmid, a bacteriophage, or an artificial chromosome. The selection of a vector will depend upon the preferred transformation technique and the target species for transformation. Accordingly, in further embodiments, a recombinant nucleic acid molecule of the invention can be comprised within a recombinant vector. The size of a vector can vary considerably depending on whether the vector comprises one or multiple expression cassettes (e.g., for molecular stacking). Thus, a vector size can range from about 3 kb to about 30 kb. Thus, in some embodiments, a vector is about 3 kb, 4 kb, 5 kb, 6 kb, 7 kb, 8 kb, 9 kb, 10 kb, 11 kb, 12 kb, 13 kb, 14 kb, 15 kb, 16 kb, 17 kb, 18 kb, 19 kb, 20 kb, 21 kb, 22 kb, 23 kb, 24 kb, 25 kb, 26 kb, 27 kb, 28 kb, 29 kb, 30 kb, or any range therein, in size. In some particular embodiments, a vector can be about 3 kb to about 10 kb in size.

A large number of vectors known in the art may be used to manipulate nucleic acids, incorporate response elements and promoters into genes, etc. For example, the insertion of nucleic acid fragments corresponding to response elements and promoters into a suitable vector can be accomplished by ligating the appropriate nucleic acid fragments into a chosen vector that has complementary cohesive termini. Alternatively, the ends of the nucleic acid molecules may be enzymatically modified or any site may be produced by ligating nucleotide sequences (linkers) to the nucleic acid termini. Such vectors may be engineered to contain sequences encoding selectable markers that provide for the selection of cells that contain the vector and/or have incorporated the nucleic acid of the vector into the cellular genome. Such markers allow identification and/or selection of host cells that incorporate and express the proteins encoded by the marker. A “recombinant” vector refers to a viral or non-viral vector that comprises one or more heterologous nucleotide sequences (i.e., transgenes). Vectors may be introduced into cells by any suitable method known in the art, including, but not limited to, transfection, electroporation, microinjection, transduction, cell fusion, DEAE dextran, calcium phosphate precipitation, lipofection (lysosome fusion), and use of a gene gun or nucleic acid vector transporter.

One aspect of the invention relates to a composition comprising the synthetic plant miRNA precursor, recombinant nucleic acid molecule, expression cassette, or vector of the invention. The composition may be a liquid or a solid that is suitable for administration to plants or plant parts as is well known in the art. A liquid composition may comprise water, saline, buffer or other solution that is suitable for use with polynucleotides. A solid composition may be a powder or other solid suitable for application to plants or plant parts.

In some aspects, a method of producing a plant that is resistant to a plant pest is provided, the method comprising: introducing into said plant or plant part a synthetic DCL-1 resistant miRNA precursor molecule of the invention, said miRNA precursor molecule comprising a guide sequence complementary to target polynucleotide or target gene in said plant pest, optionally wherein the synthetic DCL-1 resistant miRNA precursor molecule of the invention can be comprised in or encoded by a recombinant nucleic acid, an expression cassette or a vector to produce a transgenic plant or plant part, thereby producing a transgenic plant or plant part that is resistant to a plant pest.

In further aspects, a method of producing a plant that is resistant to a plant pest is provided, the method comprising: introducing into a plant cell a synthetic DCL-1 resistant miRNA precursor molecule of the invention, said miRNA precursor molecule comprising a guide sequence complementary to target polynucleotide or target gene of said plant pest, optionally wherein the synthetic DCL-1 resistant miRNA precursor molecule of the invention can be comprised in or encoded by a recombinant nucleic acid, an expression cassette or a vector to produce a transgenic plant cell; and regenerating a plant or plant part from said plant cell, thereby producing a transgenic plant or plant part that is resistant to a plant pest.

In additional aspects, a method of modulating the expression of a target polynucleotide or a target gene in a plant pest is provided, the method comprising: providing a plant produced by the methods of the invention; and exposing the plant to the plant pest under conditions wherein the plant pest takes up the synthetic plant miRNA precursor; thereby modulating the expression of a target polynucleotide or a target gene in the plant pest.

In other aspects, a method of controlling a plant pest is provided, the method comprising: providing a plant produced by the methods of the invention; and exposing the plant to the plant pest under conditions wherein the plant pest takes up the synthetic plant miRNA precursor; thereby controlling the plant pest.

In further aspects, a method of reducing damage in a plant caused by a plant pest is provided, the method comprising planting the seed of the present invention comprising the synthetic DCL-1 resistant miRNA precursor of the invention, thereby reducing damage caused by the pest to a plant grown from the seed.

In additional aspects, a method of providing a farmer with a means of controlling a plant pest is provided, the method comprising supplying to the farmer the plant of the invention.

In some aspects, the expression of the target nucleic or target gene in the plant pest can be decreased compared to a control. In other aspects, the expression of the target nucleic or target gene can be increased compared to a control. A control can include, but is not limited to, a plant pest that has not taken up or ingested a synthetic DCL-1 resistant miRNA precursor of the invention, or a control can be a plant pest that has taken up or ingested a synthetic DCL-1 resistant miRNA precursor of the invention comprising a guide strand having no complementarity to said target gene or target polynucleotide (or no complementarity to any target gene or target polynucleotide) in said plant pest.

In some aspects, a transgenic plant, plant part or plant cell can comprise two or more (e.g., 2, 3, 4, 5, 6, 7, 8, 9, 10, etc.) different synthetic DCL-1 resistant miRNA precursor molecules of the invention.

“Introducing,” in the context of a nucleotide sequence of interest (e.g., a nucleotide sequence encoding a synthetic DCL-1 resistant miRNA precursor molecule of the invention), means presenting the nucleotide sequence of interest to the plant, plant part, and/or plant cell in such a manner that the nucleotide sequence gains access to the interior of a cell. Where more than one nucleotide sequence is to be introduced these nucleotide sequences can be assembled as part of a single polynucleotide or nucleic acid construct, or as separate polynucleotide or nucleic acid constructs, and can be located on the same or different transformation vectors. Accordingly, these polynucleotides can be introduced into plant cells in a single transformation event, in separate transformation events, or, e.g., as part of a breeding protocol. Thus, for example, “introducing” can encompass transformation of an ancestor plant with a nucleotide sequence of interest followed by conventional breeding process to produce progeny comprising said nucleotide sequence of interest.

Transformation of a cell may be stable or transient. Thus, in some embodiments, a plant cell of the invention is stably transformed with a nucleotide sequence encoding a synthetic DCL-1 resistant miRNA precursor molecule of the invention. In other embodiments, a plant of the invention is transiently transformed with a nucleotide sequence encoding a synthetic DCL-1 resistant miRNA precursor molecule of the invention.

“Transient transformation” in the context of a polynucleotide means that a polynucleotide is introduced into the cell and does not integrate into the genome of the cell.

“Stable transformation” or “stably transformed,” “stably introducing,” or “stably introduced” as used herein means that a nucleic acid is introduced into a cell and integrates into the genome of the cell. As such, the integrated nucleic acid is capable of being inherited by the progeny thereof, more particularly, by the progeny of multiple successive generations. “Genome” as used herein also includes the nuclear and the plastid genome, and therefore includes integration of the nucleic acid into, for example, the chloroplast genome. Stable transformation as used herein can also refer to a transgene that is maintained extrachromasomally, for example, as a minichromosome.

Transient transformation may be detected by, for example, by an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant). Stable transformation of a cell can be detected by, for example, a Northern blot hybridization assay of RNA of the cell with nucleic acid sequences which specifically hybridize with a nucleotide sequence of a transgene introduced into a plant or other organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

Methods of introducing a nucleic acid into a plant can also comprise in vivo modification of nucleic acids, methods for which are known in the art. For example, in vivo modification can be used to insert a nucleic acid comprising, e.g., a promoter sequence into the plant genome. In a further non-limiting example, in vivo modification can be used to modify the endogenous nucleic acid itself and/or a endogenous transcription and/or translation factor associated with the endogenous nucleic acid, such that the transcription and/or translation of said endogenous nucleic acid is altered, thereby altering the expression said endogenous nucleic acid and/or in the case of nucleic acids encoding polypeptides, the production of said polypeptide.

Exemplary methods of in vivo modification include zinc finger nuclease, CRISPR-Cas, TALEN, TILLING (Targeted Induced Local Lesions IN Genomes) and/or engineered meganuclease technology.

For example, suitable methods for in vivo modification include the techniques described in Urnov et al., Nature Reviews 11:636-646 (2010)); Gao et al., Plant J. 61, 176 (2010); Li et al., Nucleic Acids Res. 39, 359 (2011); Miller et al, 29, 143-148 (2011); Christian et al, Genetics 186, 757-761 (2010)); Jiang et al., Nat. Biotechnol. 31, 233-239 (2013)); U.S. Pat. Nos. 7,897,372 and 8,021,867; U.S. Patent Publication No. 2011/0145940 and in International Patent Publication Nos. WO 2009/114321, WO 2009/134714 and WO 2010/079430; U.S. Pat. Nos. 8,795,965 and 8,771,945. For example, one or more transcription affector-like nucleases (TALEN) and/or one or more meganucleases may be used to incorporate an isolated nucleic acid comprising a promoter sequence of the invention into the plant genome. In representative embodiments, the method comprises cleaving the plant genome at a target site with a TALEN and/or a meganuclease and providing a nucleic acid that is homologous to at least a portion of the target site and further comprises a promoter sequence of the invention (optionally in operable association with a heterologous nucleotide sequence of interest), such that homologous recombination occurs and results in the insertion of the promoter sequence of the invention into the genome. Alternatively, in some embodiments, a CRISPR-Cas system can be used to specifically edit the plant genome so as to alter the expression of endogenous nucleic acids described herein. In some embodiments, a genetic modification may also be introduced using the technique of TILLING, which combines high-density mutagenesis with high-throughput screening methods. Methods for TILLING are well known in the art (McCallum, Nature Biotechnol. 18, 455-457, 2000, Stemple, Nature Rev. Genet. 5, 145-150, 2004).

As would be understood by the skilled artisan, the polynucleotides of the invention can be modified in vivo using the above described methods as well as any other method of in vivo modification known or later developed.

Thus, one or more nucleotide sequences encoding one or more synthetic DCL-1 resistant miRNA precursor molecules of the invention can be introduced into a cell by any method known to those of skill in the art. In some embodiments of the invention, transformation of a cell comprises nuclear transformation. In other embodiments, transformation of a cell comprises plastid transformation (e.g., chloroplast transformation).

Procedures for transforming plants are well known and routine in the art and are described throughout the literature. Non-limiting examples of methods for transformation of plants include transformation via bacterial-mediated nucleic acid delivery (e.g., via Agrobacteria), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

Agrobacterium-mediated transformation is a commonly used method for transforming plants, in particular, dicot plants, because of its high efficiency of transformation and because of its broad utility with many different species. Agrobacterium-mediated transformation typically involves transfer of the binary vector carrying the foreign DNA of interest to an appropriate Agrobacterium strain that may depend on the complement of vir genes carried by the host Agrobacterium strain either on a co-resident Ti plasmid or chromosomally (Uknes et al. (1993) Plant Cell 5:159-169). The transfer of the recombinant binary vector to Agrobacterium can be accomplished by a triparental mating procedure using Escherichia coli carrying the recombinant binary vector, a helper E. coli strain that carries a plasmid that is able to mobilize the recombinant binary vector to the target Agrobacterium strain. Alternatively, the recombinant binary vector can be transferred to Agrobacterium by nucleic acid transformation (Hagen & Willmitzer (1988) Nucleic Acids Res. 16:9877).

Transformation of a plant by recombinant Agrobacterium usually involves co-cultivation of the Agrobacterium with explants from the plant and follows methods well known in the art. Transformed tissue is regenerated on selection medium carrying an antibiotic or herbicide resistance marker between the binary plasmid T-DNA borders.

Another method for transforming plants, plant parts and/or plant cells involves propelling inert or biologically active particles at plant tissues and cells. See, e.g., U.S. Pat. Nos. 4,945,050; 5,036,006 and 5,100,792. Generally, this method involves propelling inert or biologically active particles at the plant cells under conditions effective to penetrate the outer surface of the cell and afford incorporation within the interior thereof. When inert particles are utilized, the vector can be introduced into the cell by coating the particles with the vector containing the nucleic acid of interest. Alternatively, a cell or cells can be surrounded by the vector so that the vector is carried into the cell by the wake of the particle. Biologically active particles (e.g., dried yeast cells, dried bacterium or a bacteriophage, each containing one or more nucleic acids sought to be introduced) also can be propelled into plant tissue.

Thus, in particular embodiments of the invention, a plant cell can be transformed by any method known in the art and as described herein and intact plants can be regenerated from these transformed cells using any of a variety of known techniques. Plant regeneration from plant cells, plant tissue culture and/or cultured protoplasts is described, for example, in Evans et al. (Handbook of Plant Cell Cultures, Vol. 1, MacMilan Publishing Co. New York (1983)); and Vasil I. R. (ed.) (Cell Culture and Somatic Cell Genetics of Plants, Acad. Press, Orlando, Vol. I (1984), and Vol. II (1986)). Methods of selecting for transformed transgenic plants, plant cells and/or plant tissue culture are routine in the art and can be employed in the methods of the invention provided herein.

Likewise, the genetic properties engineered into the transgenic seeds and plants, plant parts, and/or plant cells of the invention described above can be passed on by sexual reproduction or vegetative growth and therefore can be maintained and propagated in progeny plants. Generally, maintenance and propagation make use of known agricultural methods developed to fit specific purposes such as harvesting, sowing or tilling.

A nucleotide sequence therefore can be introduced into the plant, plant part and/or plant cell in any number of ways that are well known in the art. The methods of the invention do not depend on a particular method for introducing one or more nucleotide sequences into a plant, only that they gain access to the interior of at least one cell of the plant. Where more than one nucleotide sequence is to be introduced, they can be assembled as part of a single nucleic acid construct, or as separate nucleic acid constructs, and can be located on the same or different nucleic acid constructs. Accordingly, the nucleotide sequences can be introduced into the cell of interest in a single transformation event, in separate transformation events, or, for example, in plants, as part of a breeding protocol.

As used herein, the term “plant” may refer to any suitable plant, including, but not limited to, spermatophytes (e.g., angiosperms and gymnosperms) and embryophytes (e.g., bryophytes, ferns and fern allies). In some embodiments, a plant useful with this invention includes any monocot plant and/or any dicot plant.

Representative host plants include soybean (Glycine max), corn (Zea mays), canola (Brassica napus, Brassica rapa ssp.), alfalfa (Medicago sativa), rice (Oryza sativa), rye (Secale cereale), sorghum (Sorghum bicolor, Sorghum vulgare), sunflower (Helianthus annuus), wheat (Triticum aestivum), tobacco (Nicotiana tabacum), potato (Solanum tuberosum), peanuts (Arachis hypogaea), cotton (Gossypium hirsutum), sweet potato (Ipomoea batatas), cassava (Manihot esculenta), coffee (Coffea ssp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus carica), guava (Psidium guajava), mango (Mangifera indica), olive (Olea europaea), papaya (Carica papaya), cashew (Anacardium occidental), macadamia (Macadamia integrifolia), almond (Prunus amygdalus), sugar beets (Beta vulgaris), oats, barley, vegetables, ornamentals, and conifers.

Additional host plants of the invention are crop plants, for example, cereals and pulses, maize, wheat, potatoes, tapioca, rice, sorghum, millet, cassava, barley, pea, and other root, tuber, or seed crops or turf grasses. Important seed crops for the invention are oil-seed rape, sugar beet, maize, sunflower, soybean, and sorghum. Horticultural plants to which the invention may be applied may include lettuce, endive, and vegetable brassica including cabbage, broccoli, and cauliflower, and carnations, geraniums, petunias, and begonias. The invention may be applied to tobacco, cucurbits, carrot, strawberry, sunflower, tomato, pepper, chrysanthemum, poplar, eucalyptus, and pine. Optionally, plants of the invention include grain seeds, such as corn, wheat, barley, rice, sorghum, rye, etc. Optionally, plants of the invention include oil-seed plants. Oil seed plants include canola, cotton, soybean, safflower, sunflower, brassica, maize, alfalfa, palm, coconut, etc. Optionally, plants of the invention include leguminous plants. Leguminous plants include beans and peas. Beans include guar, locust bean, fenugreek, soybean, garden beans, cowpea, mung bean, lima bean, fava bean, lentils, chickpea, etc. Host plants useful in the invention are row crops and broadcast crops. Non-limiting examples of useful row crops are corn, soybeans, cotton, amaranth, vegetables, rice, sorghum, wheat, milo, barley, sunflower, durum, and oats. Non-limiting examples of useful broadcast crops are sunflower, millet, rice, sorghum, wheat, milo, barley, durum, and oats. Host plants useful in the invention are monocots and dicots. Non-limiting examples of useful monocots are rice, corn, wheat, palm trees, turf grasses, barley, and oats. Non-limiting examples of useful dicots are soybean, cotton, alfalfa, canola, flax, tomato, sugar beet, sunflower, potato, tobacco, corn, wheat, rice, lettuce, celery, cucumber, carrot, and cauliflower, grape, and turf grasses. Host plants useful in the invention include plants cultivated for aesthetic or olfactory benefits. Non-limiting examples include flowering plants, trees, grasses, shade plants, and flowering and non-flowering ornamental plants. Host plants useful in the invention include plants cultivated for nutritional value, fibers, wood, and industrial products.

In some particular embodiments, a plant of the invention includes, but is not limited to, a soybean plant, a sugar beet plant, a corn plant, a cotton plant, a canola plant, a sugar cane plant, a wheat plant, a rice plant or a turf grass plant. In other embodiments, a plant cell of the invention includes, but is not limited to, a soybean cell, a sugar beet cell, a corn cell, a cotton cell, a canola cell, a sugar cane cell, a wheat cell, a rice cell or the cell of a turf grass.

As used herein, the term “plant part” includes but is not limited to embryos, pollen, ovules, seeds, leaves, flowers, branches, fruit, kernels, ears, cobs, husks, stalks, roots, root tips, anthers, plant cells including plant cells that are intact in plants and/or parts of plants, plant protoplasts, plant tissues, plant cell tissue cultures, plant calli, plant clumps, and the like. Further, as used herein, “plant cell” refers to a structural and physiological unit of the plant, which comprises a cell wall and also may refer to a protoplast. A plant cell of the invention can be in the form of an isolated single cell or can be a cultured cell or can be a part of a higher-organized unit such as, for example, a plant tissue or a plant organ. A “protoplast” is an isolated plant cell without a cell wall or with only parts of the cell wall. Thus, in some embodiments of the invention, a transgenic cell comprising a nucleic acid molecule and/or nucleotide sequence of the invention is a cell of any plant or plant part including, but not limited to, a root cell, a leaf cell, a tissue culture cell, a seed cell, a flower cell, a fruit cell, a pollen cell, and the like. In some aspects of the invention, the plant part can be a plant germplasm. In some aspects, a plant cell can be non-propagating plant cell that does not regenerate into a plant.

“Plant cell culture” means cultures of plant units such as, for example, protoplasts, cell culture cells, cells in plant tissues, pollen, pollen tubes, ovules, embryo sacs, zygotes and embryos at various stages of development. In some embodiments of the invention, a transgenic tissue culture or transgenic plant cell culture is provided, wherein the transgenic tissue or cell culture comprises a nucleic acid molecule/nucleotide sequence of the invention.

As used herein, a “plant organ” is a distinct and visibly structured and differentiated part of a plant such as a root, stem, leaf, flower bud, or embryo.

“Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

A further aspect of the invention provides transformed non-human host cells and transformed non-human organisms comprising the transformed non-human cells, wherein the transformed cells and transformed organisms comprise a synthetic DCL-1 resistant miRNA precursor molecule of the invention. In some embodiments, the transformed non-human host cell includes but is not limited to a transformed fungal cell (e.g., a transformed yeast cell), a transformed insect cell, a transformed bacterial cell, and/or a transformed plant cell. Thus, in some embodiments, the transformed non-human organism comprising the transformed non-human host cell includes, but is not limited to, a transformed yeast, a transformed insect, a transformed bacterium, and/or a transformed plant.

In some aspects, the invention provides plants, plant parts, and/or plant cells produced by the methods of the invention. In representative embodiments, the invention provides a seed from a plant of the invention comprising in its genome a synthetic DCL-1 resistant miRNA precursor molecule of the invention and a plant grown from said seed. Additional aspects of the invention include a product harvested from the plants and/or parts thereof of the invention, as well as a post-harvest product produced from said harvested product. A harvested product can be a whole plant or any plant part, as described herein, wherein said harvested product comprises a nucleotide sequence encoding at least one of the miRNA precursor molecules of the invention. Thus, in some embodiments, non-limiting examples of a harvested product include a seed, a fruit, a flower or part thereof (e.g., an anther, a stigma, and the like), a leaf, a stem, and the like. In other embodiments, a post-harvest product includes, but is not limited to, a flour, meal, oil, starch, cereal, and the like produced from a harvested seed of the invention, wherein said seed comprises in its genome a nucleotide sequence encoding at least one of the miRNA precursor molecules of the invention.

In some embodiments, the invention further provides a plant crop comprising a plurality of transgenic plants of the invention planted together in, for example, an agricultural field, a golf course, a residential lawn, a road side, an athletic field, and/or a recreational field.

In one aspect of the invention, the synthetic plant miRNA precursors of the invention can be provided to a plant by means other than creation of a transgenic plant. In some embodiments, the invention relates to a method of reducing damage in a plant caused by a plant pest, the method comprising applying to the plant the synthetic plant miRNA precursor molecule of the invention, thereby reducing damage caused by the pest.

The synthetic plant miRNA precursors and compositions thereof of the invention can be applied to the surface of a plant or plant part, including but not limited to, seed, leaves, flowers, stems, tubers, roots, and the like. In some embodiments, the application is carried out by soaking seeds or chemically coating seeds with the synthetic plant miRNA precursors and compositions. In some embodiments, the synthetic plant miRNA precursors and compositions of the invention are delivered orally to a plant pest, e.g., an insect or nematode, wherein the plant pest ingests one or more parts of a plant to which a composition comprising the synthetic plant miRNA precursors of the invention has been applied. Applying the compositions of the invention to a plant can be done using any method known to those of skill in the art for applying compounds, compositions, formulations and the like to plant surfaces. Some non-limiting examples of applying include spraying, dusting, sprinkling, scattering, misting, atomizing, broadcasting, soaking, soil injection, soil incorporation, drenching (e.g., root, soil treatment), dipping, pouring, coating, leaf or stem infiltration, side dressing or seed treatment, and the like, and combinations thereof. In certain embodiments, the application may comprise grafting of plant tissue, wherein a trait phenotype is acquired by mobilization of RNA molecules from stock tissue to scion tissue. These and other procedures for applying a compound(s), composition(s) or formulation(s) to a plant or part thereof are well-known to those of skill in the art.

The invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the invention.

### EXAMPLES

### Example 1

### Methods for Designing DCL-1 Resistant miRNA Precursors

Natural endogenous miRNA precursors were analyzed to identify all the possible features of plant miRNA precursors that affect processing by DCL-1. These features were classified into 3 categories: physical features, sequence features, and structural features. All of these features are shown to be valuable for synthetic precursor design.

Step 1. Modifications of Structural Folding Consensus of Plant miRNA Precursor to Generate DCL-1 Resistant Precursor

Because any synthetic design requires a backbone as a platform, a previously identified DCL-1 preferred precursor structure (dp0019; SEQ ID NO: 1) was selected as the starting point. dp0019 was developed from a composite of the following naturally occurring precursors: Oryza sativa miR159a, Sorghum bicolor miR159, Saccharum officinarum miR159a, Saccharum officinarum miR159b, Zea mays miR159, Zea mays miR159b, Triticum aestivum miR159b, Zea mays miR159f, Zea mays miR159g, Zea mays miR159i, Zea mays miR159j, and Saccharum ssp miR159a.

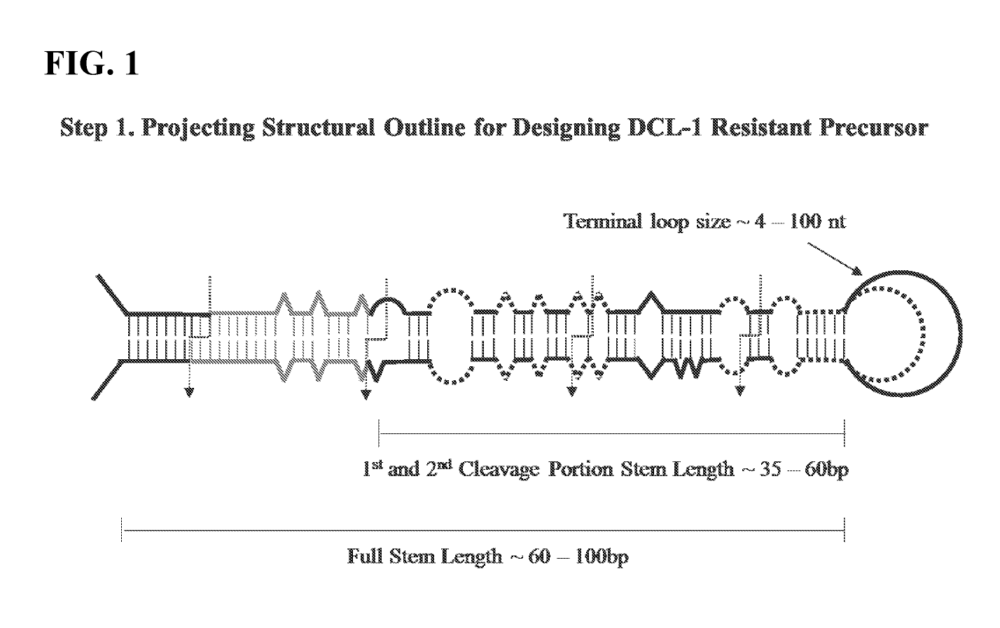

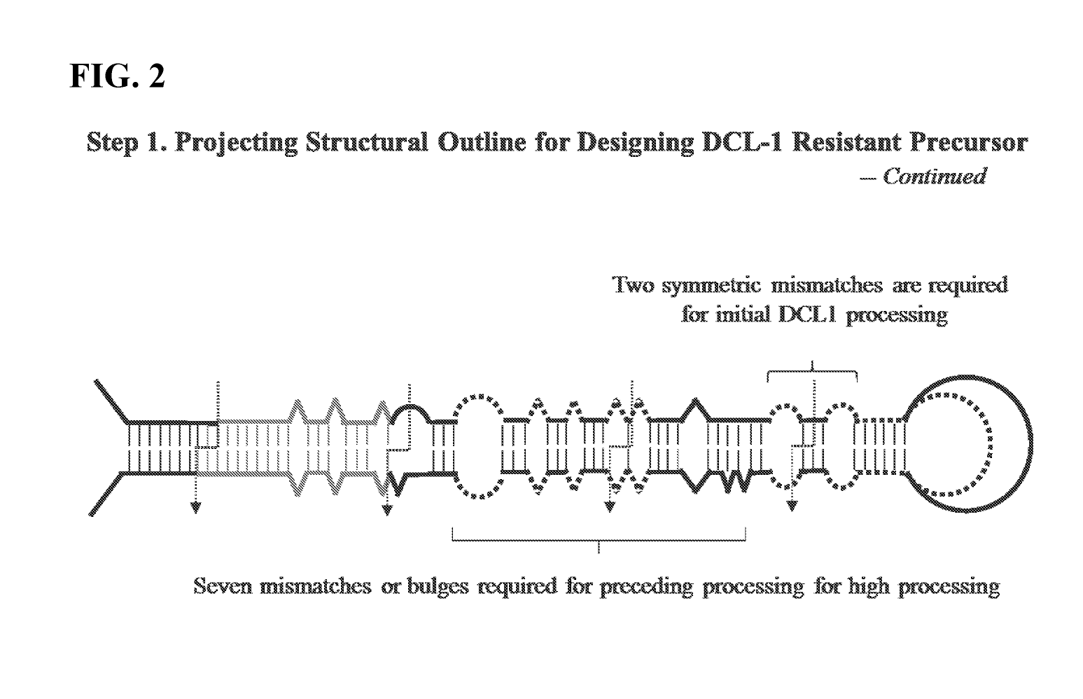

Also, because the synthetic design needs to be recognized and processed by pest endogenous Dicer processing machinery, and based on the study of natural plant miRNA precursor processing pathways, the mechanism of loop to base was chosen as a model pathway for the design. As shown in FIG. 1, the DCL-1 resistant synthetic precursors were designed to preserve the length for the loop to base mechanism. A structural outline of DCL-1 resistant precursors was designed with modification of structural motifs important for the initial recognition by maize DCL-1, and thereby preventing successive processing following the initial recognition and initial processing events. As indicated in FIG. 1, examples of these changes have included the 2 symmetric mismatches which cover the 5′ end of the first cleavage site, changing the 3′ end of the first cleavage site, one mismatch proximal to the left side of the miRNA/miRNA* duplex, etc. (FIG. 2).

### Step 2. Composition of Sequence Feature for the Design According to Structure

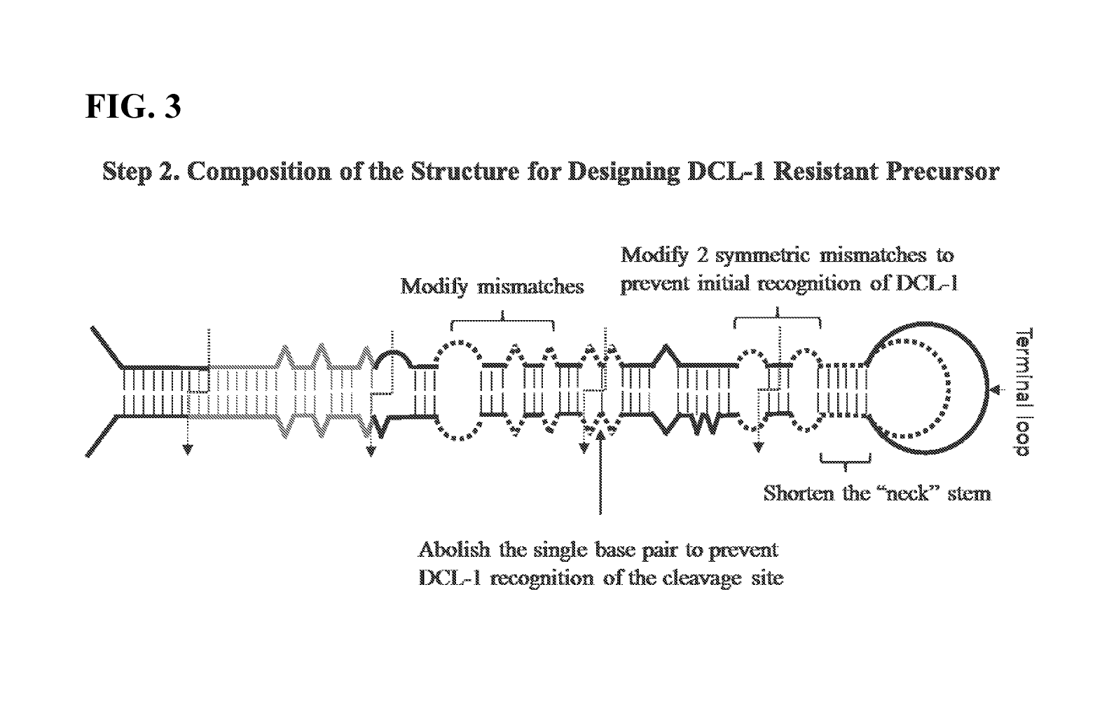

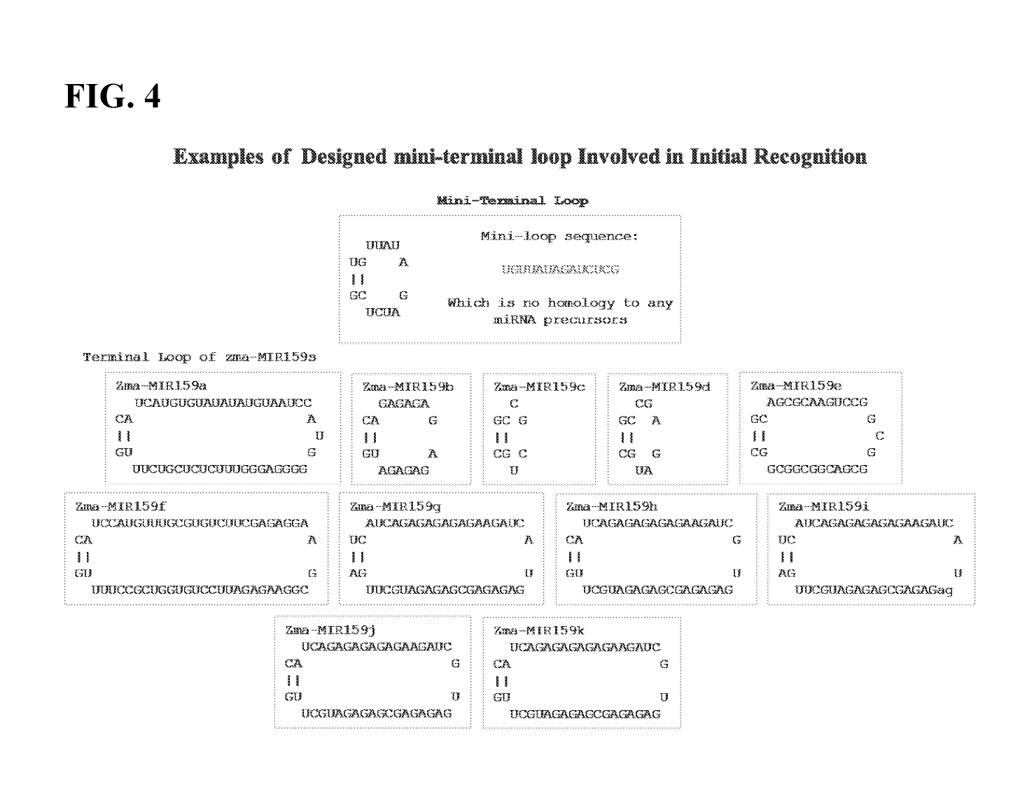

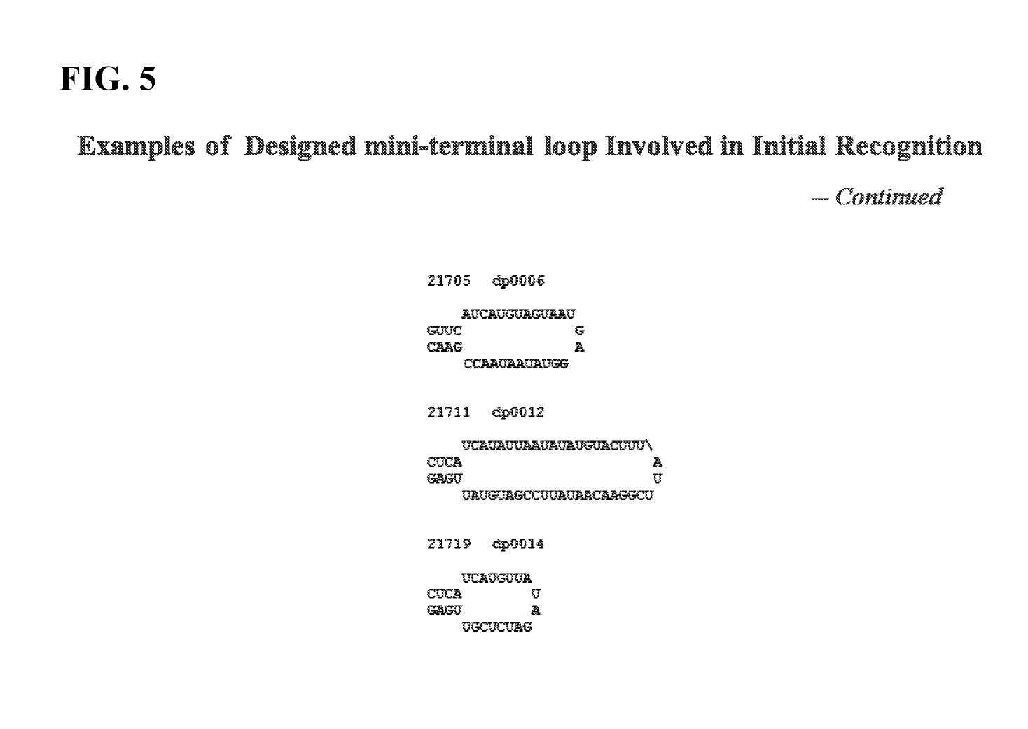

Based on the structural outline shown in FIG. 1 and FIG. 2, the next step was to fill in the RNA nucleotide sequence. As shown in FIG. 3, five important classes of sequence were identified, including the terminal loop, neck stem, important mismatches, bulges, and DCL-1 cleavage sites. These five regions were the focus for changes and modifications as pointed out in FIG. 3.

### 1. Terminal Loop

The terminal loop is an important structural element for any hairpin-based RNAi design. In the present invention, a mini-loop was designed (SEQ ID NO. 13) Importantly, this mini-loop enables successful recognition and processing of synthetic miRNA precursors not only in transgenic maize, but also in the tested western corn rootworm (Diabrotica virgifera virgifera). The designed synthetic loop is totally different from a list of endogenous terminal loops of the maize zma-MIR159/319 family. A number of terminal loops for DCL-1 resistant precursors were designed (SEQ ID NO. 14, SEQ ID NO. 15, SEQ ID NO. 16).

### 2. Modification of Big Symmetric Mismatches in Proximal to Terminal Loop for Preventing the Initial Recognition by DCL-1 in Transgenic Maize

To initiate the mechanism of loop to base processing, an important step is the initial recognition by DCL-1. In the present invention, the sequence context of the identified structures of symmetric mismatches in the synthetic precursors were modified. Some precursors retained symmetric mismatches but the sequence was changed (e.g., a change in the sequence GUUU). In one precursor the symmetric mismatches became asymmetric mismatches (bulges). The resultant mismatches no longer share sequence identity with endogenous zma-MIR159 or highly active dp0019. Some of these designs impaired or abolished DCL-1 recognition of the precursor in transgenic maize plants.

### 3. Design of Bulges and Mismatches to Prevent DCL-1 Processing

Bulges and mismatches are fundamental due to their roles in either fingerprint signaling for processing machinery recognition or cleavage sites of DCL-1. Therefore, the changes of some important bulges could substantially avoid DCL-1 recognition and continuing successive processing. Based on this reasoning, a number of modified regions were designed for the present invention (FIG. 7).

### 4. Modifications of Cleavage Sites by DCL-1 LED to Prevent DCL-1 Resistance

Cleavage site features either based on sequence or structure play important roles in DCL-1 processing efficiency. Thus, changes were introduced in order to block DCL-1 recognition and/or cleavage efficacy. A variety of changes in sequence and structural features for loop to base processing mechanism were identified and successfully applied in the current designs.

### 5. Changes of the Length of Stem Segment (Neck) Adjunct to Terminal Loop LED to Failure of DCL-1 Recognition

In General, Dicer Processing Follows a Ruler to Measure not Only the Distance Between the two cleavage sites, but also to measure the locations of the cleavage sites (MacRae, et al. Nature 14:934 (2007)). This measurement was advantageously used in implementation for the design of this invention. For example, shortening the length of the neck affected DCL-1 recognition and its initial cleavage, resulting in DCL-1 resistant precursors in maize transformants.

### Step 3. Validation by Folding Shape and Folding Energy Feature Analysis for the Design

Because the purpose of the designed DCL-1 resistant precursors in transgenic plants is to become a preferred precursor for invading pests based on RNA structure, analysis of RNA folding of the DCL-1 resistant synthetic precursors is useful. During the design of precursors, folding assessments were carried out for each step. In the end, all of the designed DCL-1 resistant precursors retained the typical miRNA precursor folding.

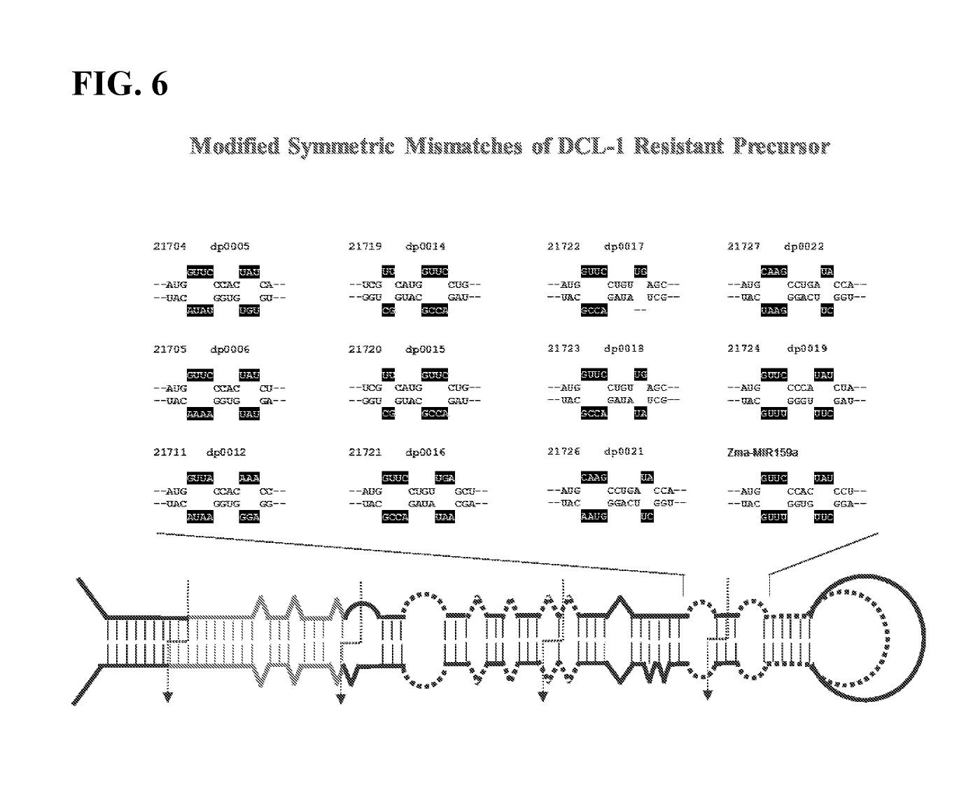

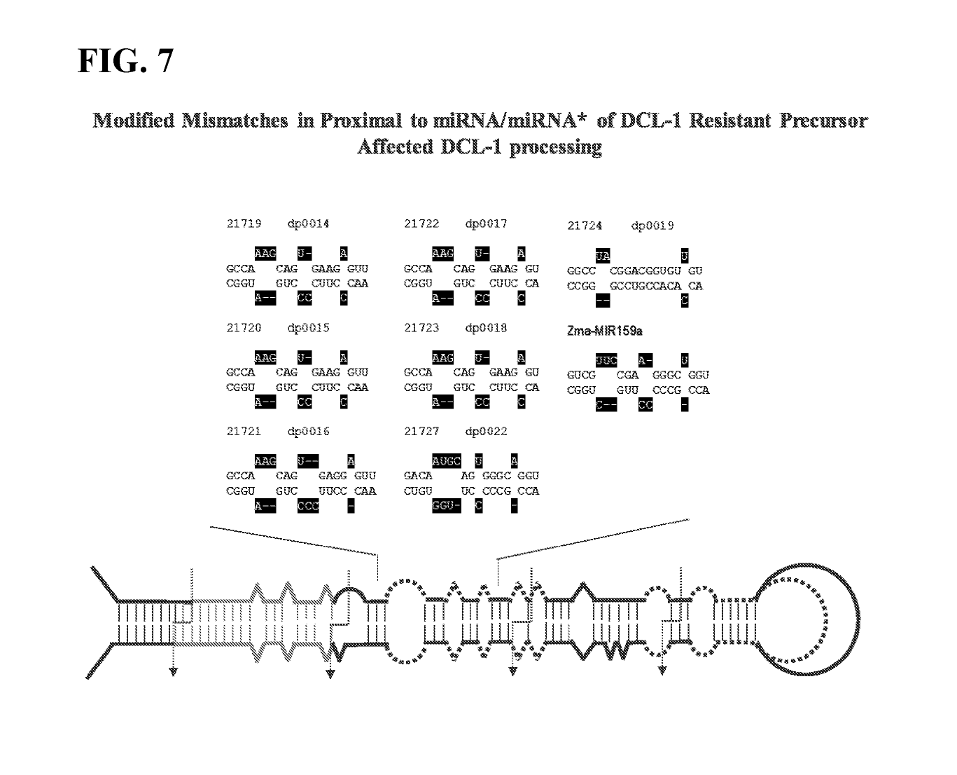

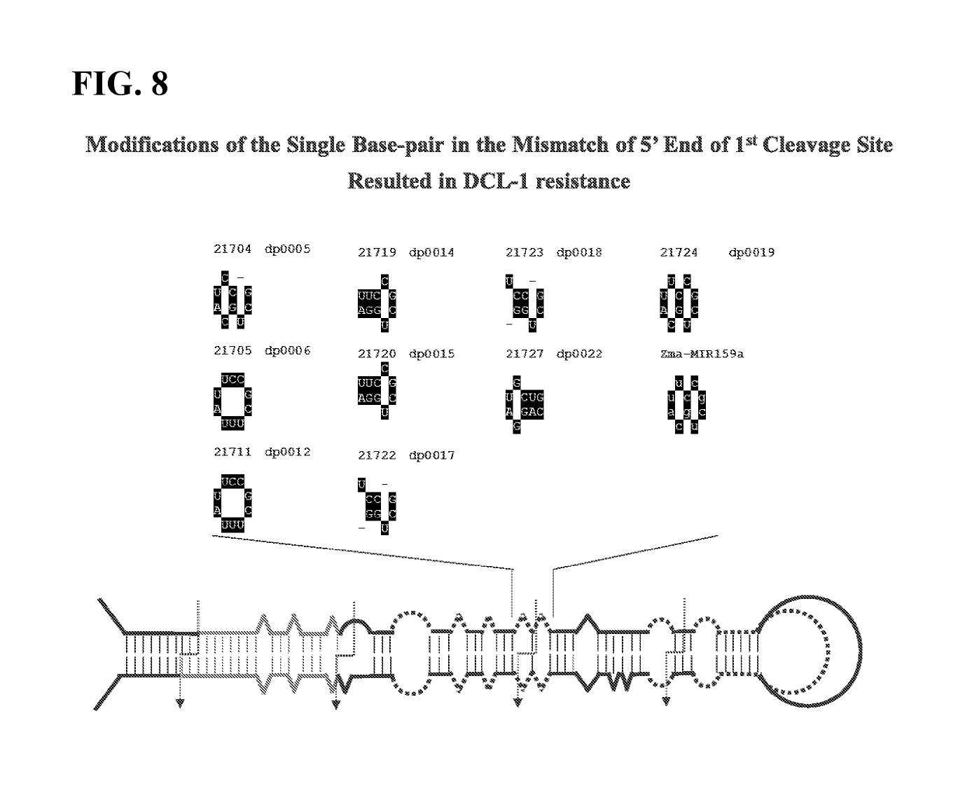

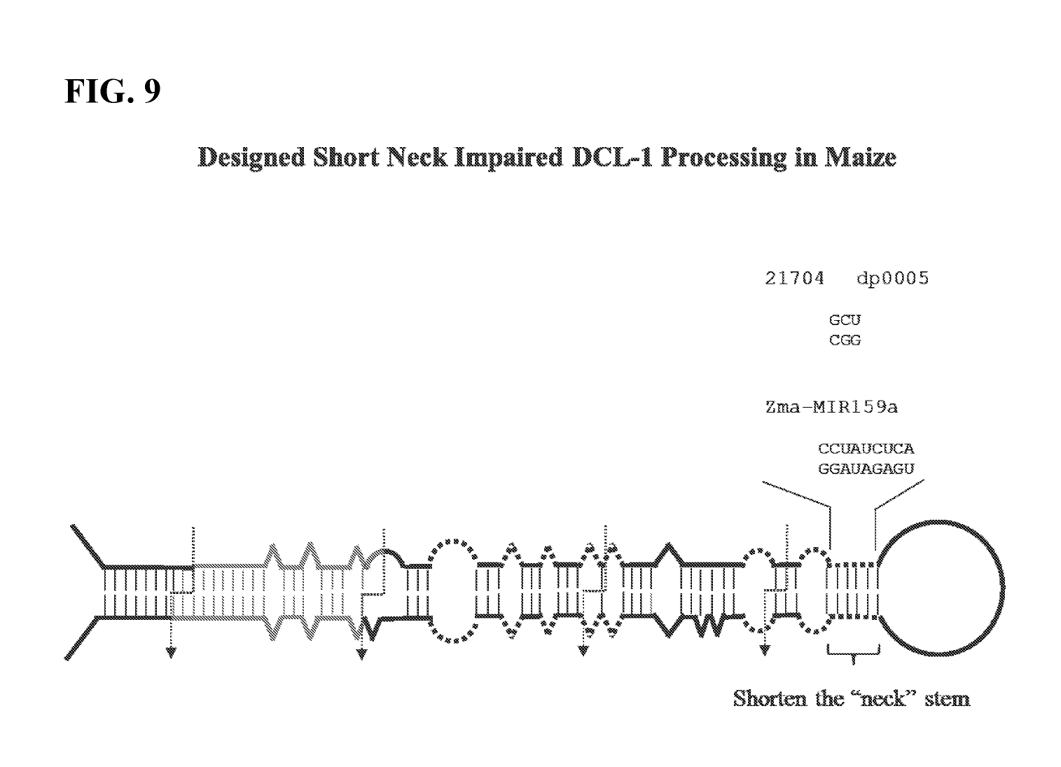

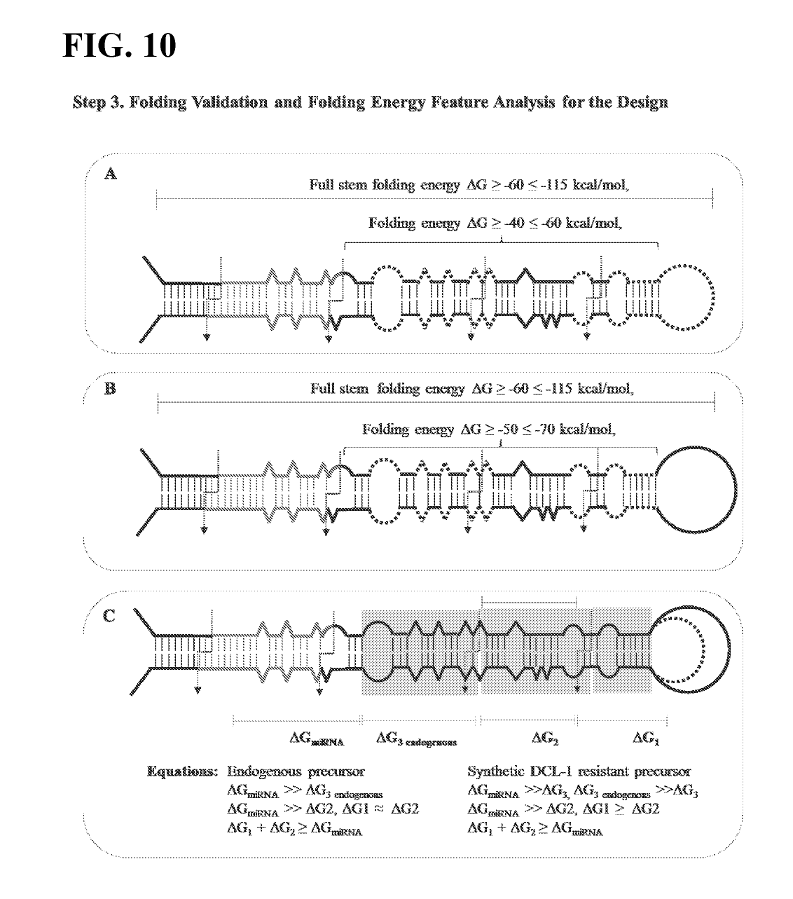

In general, a thermal dynamics description represents the RNA folding stability, which is provided in the output by an RNA folding program (mfold) during the folding process. It worth noting that one of the novelties found during the present studies is that DCL-1 recognition is sensitive to folding energy, a measurement of precursor RNA folding stability, which is also very important to direct the recognition by DCL-1 and its associated processing machinery. Overall, the designed DCL-1-resistant precursors have a relatively higher folding energy than endogenous precursors. In addition, this invention has discovered that precursor folding energy is related at least in part to terminal loop size. The ΔG of mini-loop-based DCL-1 resistant precursors is approximately in the range between −70 and −80 kcal/mol (FIG. 10A), which is higher than DCL-1 preferred precursors such as dp0019 (ΔG=−111.40 kcal/mol) and large-loop-based DCL-1 resistant precursors. Large loop-based DCL-1 resistant precursors are comparable to endogenous precursors in stability (FIG. 10B). More detailed calculations at segmental levels are presented in FIG. 10C, analyzing segments that are no longer cleaved or exhibit reduced cleavage by DCL-1.

This invention comprises an algorithm or method of designing DCL-1 resistant synthetic precursors delivering target specific silencing activity in a cross-species manner. In one embodiment, three steps were identified for the design of precursors after thorough analysis and understanding of sequences and structures of selected endogenous miRNA precursors.

In one particular embodiment, the first step is acquisition of structure and its structural features, importantly, during which all the most conserved structure elements (such as mismatches, bulges severed as cleavage sites, etc.) are used to generate a blueprint for designing. At this step, some fundamental sequences and structure elements should be considered for changes in the second step.

In a further embodiment, the second step is filling the structural backbone with sequences. At this step, the most important sequence elements are used. Then some precise modifications are introduced to these important sequences and structural features, so that DCL-1 recognition and cleavage become defective.

In yet a further embodiment, the third step is folding shape and folding energy examination. At this step, the folding shape must be retained to be miRNA precursor-like and its individual folding energy should agree with the folding energy range for a decrease or elimination of processing by DCL-1.

### Example 2

### Examples of Designed Synthetic Precursors

Out of a total of 35 designed synthetic precursors, 11 were DCL-1 resistant precursors as verified by maize transformation. The nucleotide sequence and predicted structure of the resistant precursors are shown in Table 1.

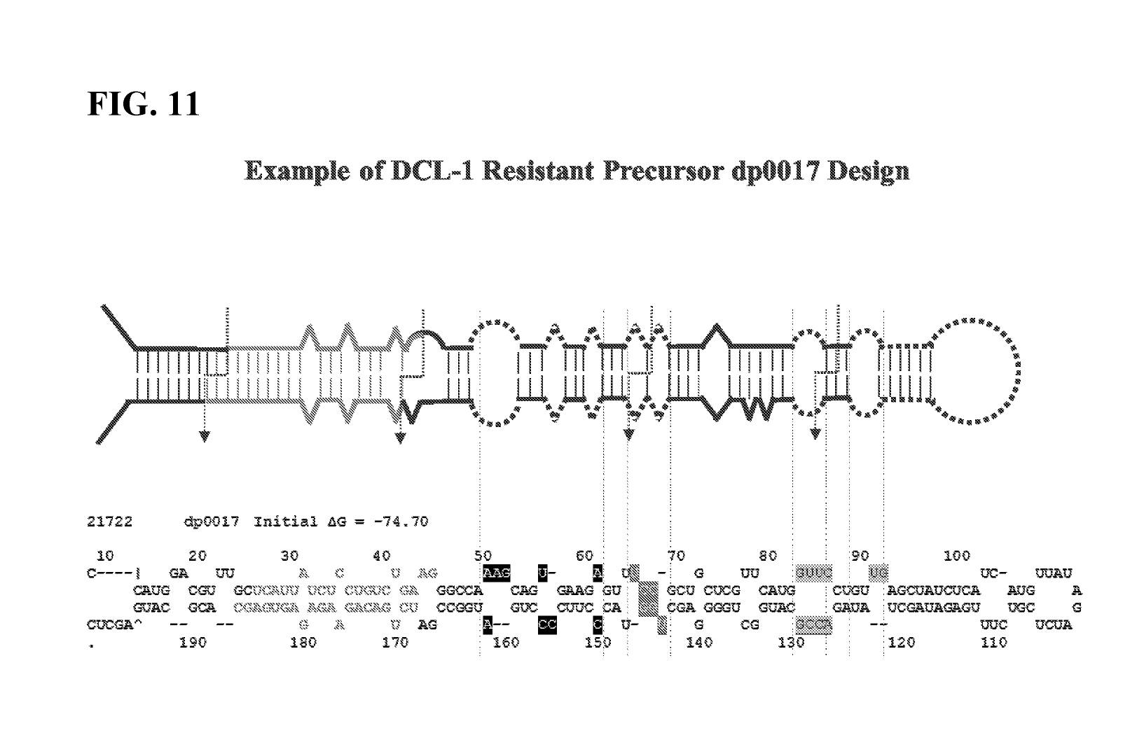

Using the requirements of sequence and structural feature as shown in FIG. 2 and FIG. 3, the design of dp0017 using the following procedure is provided as an example. First, the MIR159 family from monocot plants was chosen as a design template for synthetic precursor design due to its high expression and its carefully studied mechanism in Arabidopsis (Nicola et al., EMBO J. 8:3646-3656 (2009)). The theory was if the precursor becomes DCL-1 resistant, the precursor transcript RNA should densely accumulate in transgenic plants and thus it would be very valuable for high dose RNAi delivery in cross-species manner to plant pests, for example, transgenic maize conferring resistance to western corn rootworm. The dp0017 precursor is shown in FIG. 11.

It was decided that the structural backbone should keep the 7 small bulges and mismatches followed by 2 mismatches proximal to the terminal loop. During the designing the sequence context of the 2 mismatches was modified so that they became a mismatch and a bulge and the resultant mismatch was modified from the original mismatch GUUU to GCCA. Part of the first cleavage fragment was modified to convert the single base pair to two base pairs and convert the mismatches to bulges. Additionally, some of the mismatches and bulges in the second cleavage fragment were changed as shown in FIG. 11. All of these changes and modifications are expected to affect DCL-1 recognition and cleavage.

After composing the sequence modifications, mfold examination of the dp0017 sequence was performed for folding profiling. The initial ΔG is −74.70 kcal/mol, much higher than the DCL-1 preferred dp0019 (−111.40 kcal/mol) and endogenous zma-MIR159 (−104.80 kcal/mol), suggesting dp0017 precursor RNA folding stability is out of the scope of DCL-1 recognition in maize because of such low folding stability.

As an example of the effect of individual modifications on resistance of precursors to DCL-1, a comparison was made of the sequences of dp0017 (SEQ ID NO. 9) and dp0018 (SEQ ID NO. 10). The two precursor sequences differ only in the presence of a 2 nucleotide bulge in structure C on the 3′ side of the first cleavage site in dp0017 compared to a 2 nucleotide mismatch in structure C on the 3′ side of the first cleavage site in dp0018. Despite the small difference in structure, dp0017 is completely resistant to DCL-1 processing while dp0018 is partially resistant to DCL-1 processing.

### Example 3

### Validation of Maize Transformation

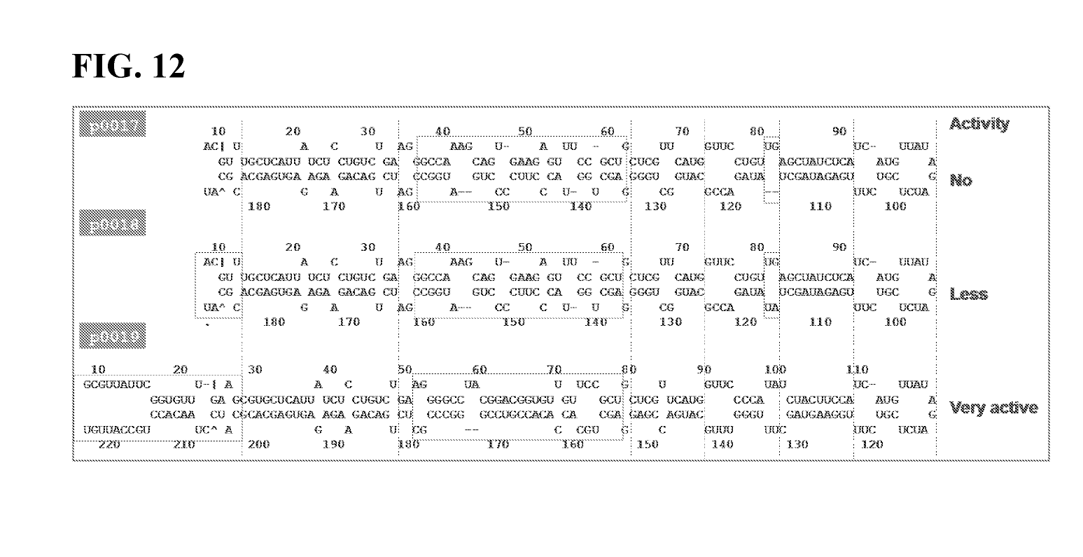

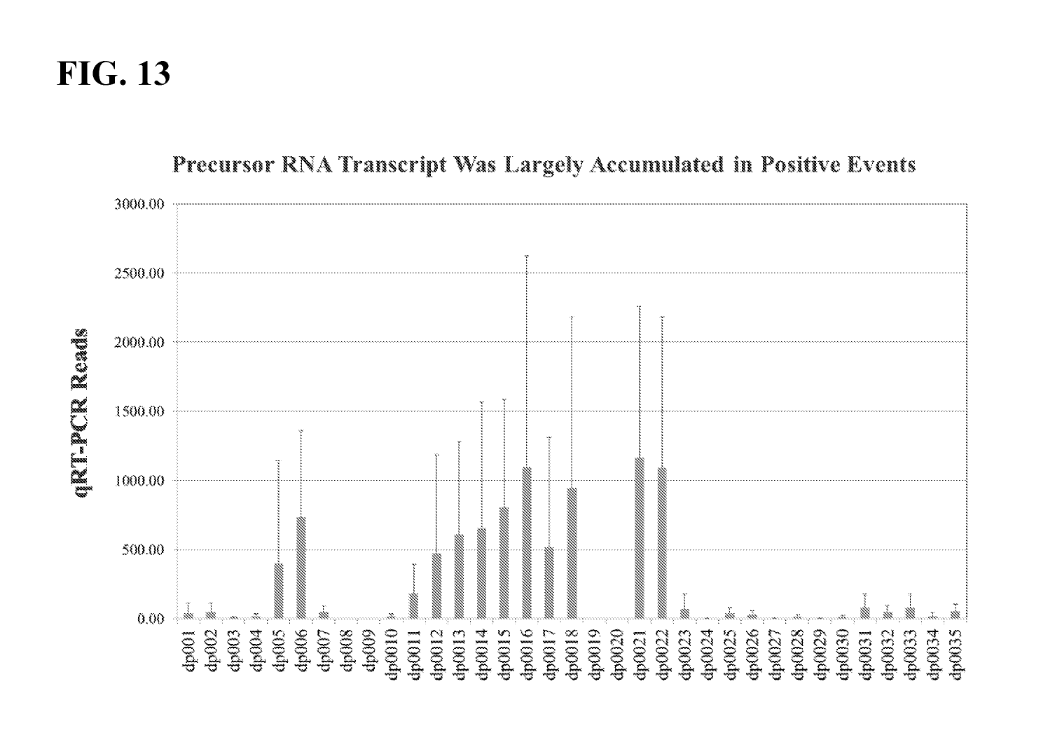

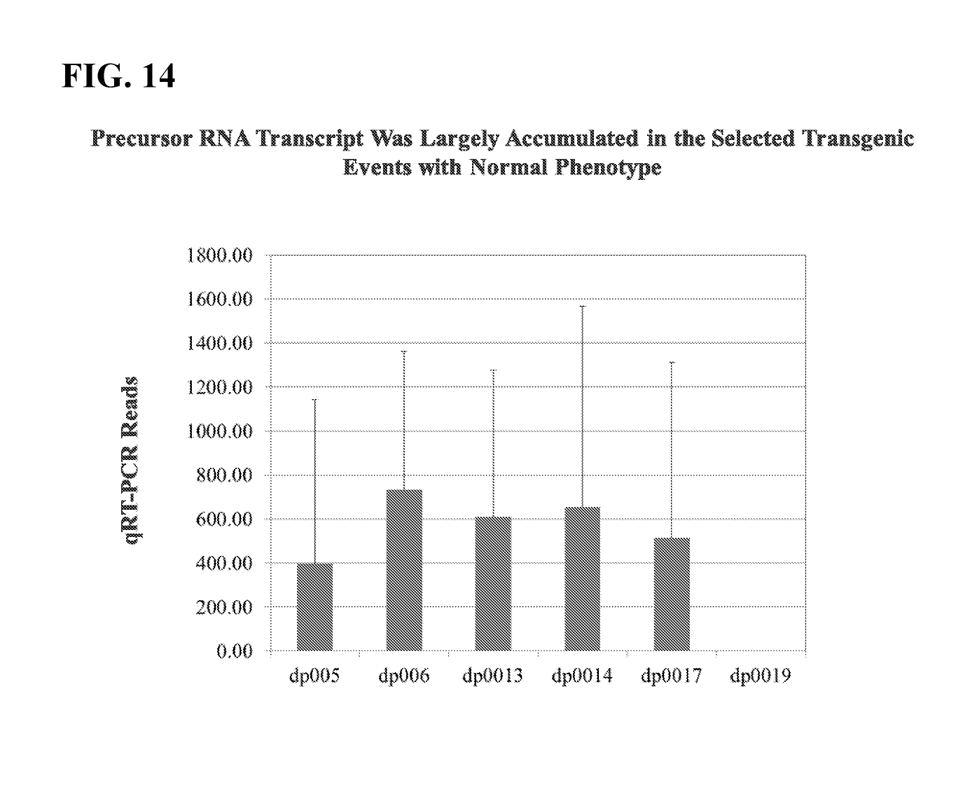

The 35 full length synthetic precursors were analyzed in transgenic maize plants individually harboring the precursors by qRT-PCR (quantitative reverse transcription-polymerase chain reaction) screening and phenotype observation. The qRT-PCR results showed very high accumulations of precursor in these plants transformed with 11 constructs. As shown in FIG. 13, the positive constructs originated from synthetic precursors dp005, dp006, dp0011, dp0013, dp0014, dp0015, dp0016, dp0017, dp0018, dp0021, and dp0022. Importantly, the transgenic plants remained normal phenotype regardless of the high accumulation of these synthetic precursor RNA molecules. In comparison, phytoxicity was observed in some plants, such as those expressing dp0016, dp0021, dp0022i, presumably due to overly high accumulation of the synthetic precursors. To compromise between the expressed RNA transcript level of DCL-1 resistant precursors and phytoxicity, we chose dp005, dp006, dp0013, dp0014, and dp0017 as these plants had a normal phenotype (expression levels shown in FIG. 14), strongly demonstrating the successes of the DCL-1 resistant synthetic precursor design.

### Example 4

### Validation of Cross-Species Functionality Using Western Corn Rootworm Bioassay

The tentative selected synthetic precursors are resistant to maize DCL-1 cleavage verified from stable maize transformation as shown in FIG. 13 and FIG. 14. The next question was whether these precursors can be processed by pest Dicer upon uptake, i.e., whether these synthetic precursors could deliver target gene silencing in pest after pest Dicer cleavage. A western corn rootworm (Diabrotica virgifera virgifera) RNAi bioassay system was developed for the evaluation. Among the synthetic precursors dp005, dp006, dp0013, dp0014, and dp0017 shown to be effective in FIG. 13, dp005 was randomly chosen as a backbone. The western corn rootworm histone-4 (His4) gene was used as a target gene for the test. High scored siRNAs were predicted by using the sfold program. From the pool of high scored siRNAs, sixteen siRNAs were chosen and inserted into dp005, which serves as a carrier to deliver the siRNA. After in vitro transcription and purification, these 16 His4 siRNA precursors were subjected to the western corn rootworm bioassay using neonate larvae. The mortality was calculated according to the designated time course.

### Insect

Western corn rootworm (WCR) (Diabrotica virgifera virgifera) eggs were purchased from French Agricultural Research, In. (Lamberton, Minn.). After receipt, eggs were hatched at 28° C. The neonate were employed as testing insects.

### Precursor RNA Preparation

DNA templates were released by PCR and purified by phenol:chloroform extraction and ethanol precipitation. In vitro transcription was carried out using 1 mg PCR product using the AmpliScribe™ T7 High Yield Transcription Manufacture Kit (Epicentre, Wis.). The synthesized dsRNA was purified by phenol:chloroform and ethanol precipitation, dissolved in water, quantified and stored at −80° C.

### Artificial Diet Preparation and Feeding Assay

300 ml diet contained 6 g agarose; 16.35 g raw wheat germ; 23 g sucrose; 4.6 g casein; 4 g cellulose; 4.2 g wessen salts; 0.3 g methyl paraben; 18 mg cholesterol; 2.7 g Vanderzant's Vitamin mix; and 192 mg sorbic acid. After autoclaving, nystatin, cefotaxime, streptomycin, and spectinomycin were added to a final concentration of 0.17 mg/ml, 0.625 mg/ml, 0.375 mg/ml, and 0.375 mg/ml, respectively. Precursor RNA solution was overlaid onto the surface of diet in each well of a 128-well plate at 100 ng/cm². Individual WCR larvae were accommodated onto the diet afterwards. Mortality was recorded under a microscope daily.

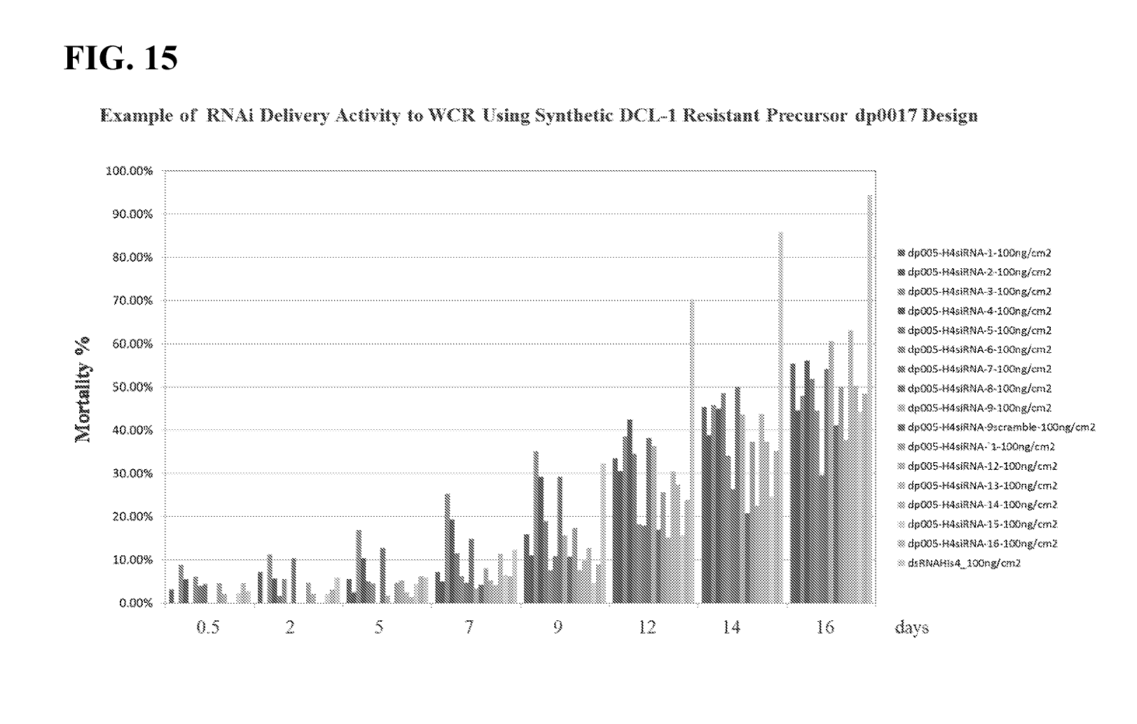

As shown in FIG. 15, visible mortality could be seen in 5 days from dp005-His4siRNA-3 and dp005-His4siRNA-4, even higher than full length His dsRNA (double-stranded RNA) after 10 days. After 12 days, the mortality from dp005-His4siRNA-8 increased very fast. The mortality rate for the scrambled siRNA of His4siRNA-9 remained at about 10% at 9 days. At 14 days, the scrambled siRNA produced only about 20% mortality, which was much lower than the non-scrambled siRNA His4siRNA-9 (about 45%). All of these observations strongly indicated that the synthetic DCL-1 resistant precursor could be recognized and processed by western corn rootworm Dicer in gut cells, enabling delivery of effective amounts of RNAi in the body during the assay as indicated by the observed mortality.

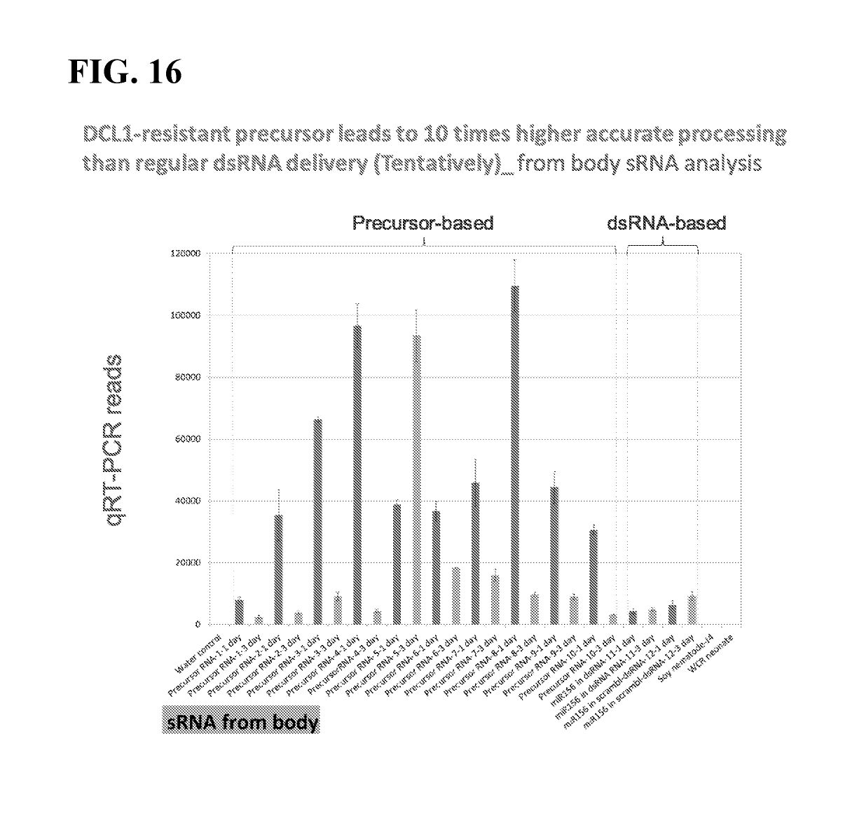

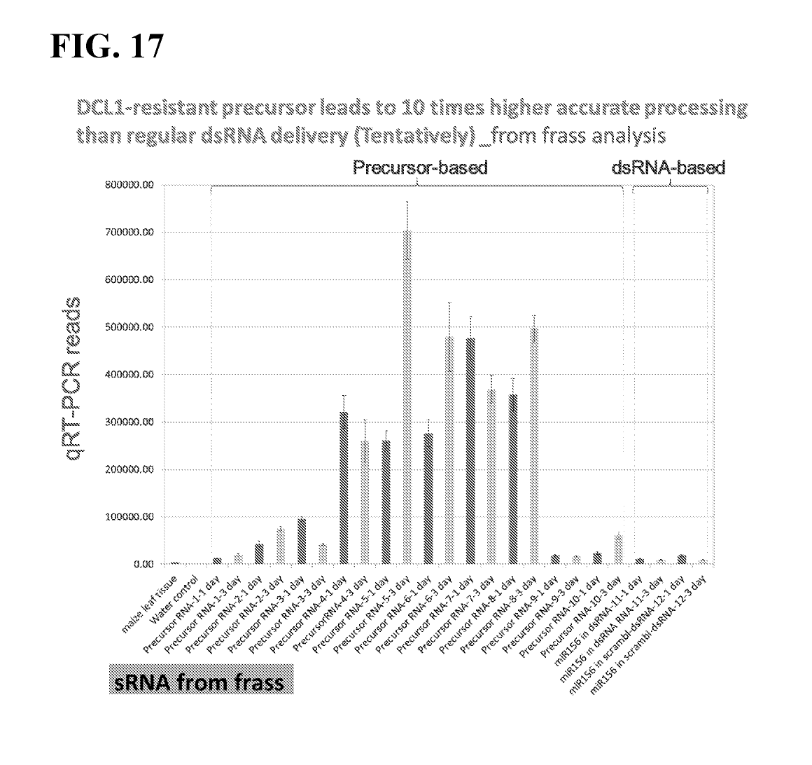

Moreover, qRT-PCR detection showed that these synthetic DC1-1 resistant precursors are recognizable to western corn rootworm Dicer during the feeding assay as displayed in FIG. 16 and FIG. 17. FIG. 16 shows a qRT-PCR analysis of miRNA processing dynamics between 24 hrs vs. 48 hrs using small RNA preparations from larvae bodies. The data indicate that these miRNAs are processed from the precursor by Dicer in the body. The blue bar indicates 24 hrs, the orange bar is 48 hrs. Comparing the two colored bars, all of the precursors (except dp0017) showed robust processing in 24 hrs. dp0017 showed higher processing in 48 hrs.

FIG. 17 shows a qRT-PCR analysis of miRNA processing out of DC1-1 resistant precursors using miRNAs extracted from frass. It shows miRNA processing dynamics between 24 hrs vs. 48 hrs. The high level of processed miRNAs in frass may be due to secretion of miRNA. Very importantly, the processing of these precursors is much more robust than the conventional dsRNA-based delivery approach.

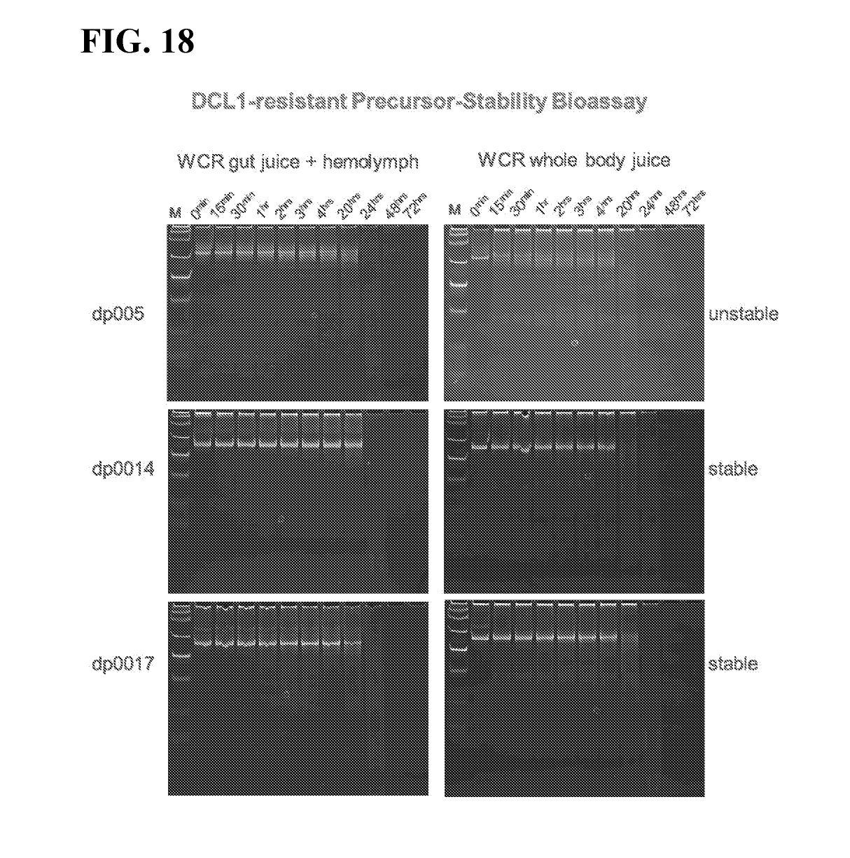

In addition, the stability of these synthetic DCL1-resistant precursor RNAs was tested in the ingestion pathway of western corn rootworm. FIG. 18 shows precursor stability assays using western corn rootworm body fluids, either gut juice mixed with hemolyph or body juice. Experiments were carried out at room temperature and aliquots taken at the indicated time points for resolution on a 1.2% agarose gel. The results demonstrated that some of the precursors are very stable (dp0014 and dp0017 vs. dp005) as shown in FIG. 18, strongly suggesting the potential value for future applications not only for a transgenic plant approach, but also for a spray RNAi approach.

This invention comprises a method for designing valuable synthetic precursors for cross-species RNAi delivery. The DCL-1 resistant designs have substantially resolved the prevalent issue of RNAi dose for cross-species RNAi delivery, providing a unique method for delivering target gene-specific RNAi to plant pests.

The foregoing is illustrative of the invention, and is not to be construed as limiting thereof. The invention is defined by the following claims, with equivalents of the claims to be included therein.

1. A synthetic plant microRNA (miRNA) precursor comprising four DCL-1 cleavage sites, the precursor comprising the following structures in loop-to-base order:
A. a terminal loop;
B. a neck stem;
C. a pair of mismatches and the flanking nucleotides surrounding a first cleavage site;
D. a pair of mismatches and/or bulges and the flanking nucleotides surrounding a second cleavage site; and/or
E. a series of mismatches and/or bulges and the flanking nucleotides between the second cleavage site and a third cleavage site;
wherein the precursor is modified at one or more of structures A, B, C, D, and E to be resistant to cleavage by a plant Dicer-like protein-1 (DCL-1) but susceptible to cleavage by Dicer or a Dicer-like protein of a plant pest.

2. The synthetic plant miRNA precursor of claim 1, wherein the modification at one or more of structures A, B, C, D, and E is a change in sequence, length, symmetry, or a combination thereof.

3. The synthetic plant miRNA precursor of claim 1, wherein the modification is made to a wild-type plant miRNA precursor sequence.

4. The synthetic plant miRNA precursor of claim 1, wherein the modification is made to a consensus plant miRNA precursor sequence.

5. The synthetic plant miRNA precursor of claim 1, wherein the modification comprises a change in the length of the terminal loop.

6. The synthetic plant miRNA precursor of claim 5, wherein the terminal loop is modified to a length of about 10 to about 50 nucleotides.

7. The synthetic plant miRNA precursor of claim 1, wherein the modification comprises a change in the length of the neck stem.

8. The synthetic plant miRNA precursor of claim 7, wherein the neck stem is shortened by 1-10 nucleotide pairs relative to the wild-type or consensus sequence.

9. The synthetic plant miRNA precursor of claim 1, wherein the modification comprises a change in the sequence, length, symmetry, or a combination thereof in one or both mismatches of the pair of mismatches surrounding the first cleavage site.

10. The synthetic plant miRNA precursor of claim 9, wherein the modification comprises changing the nucleotide sequence of one or both mismatches.

11. The synthetic plant miRNA precursor of claim 9, wherein the modification comprises shortening the length of one or both mismatches.

12. The synthetic plant miRNA precursor of claim 9, wherein the modification comprises deleting one or more nucleotides in one or both mismatches to create one or more bulges.

13. The synthetic plant miRNA precursor of claim 1, wherein the modification comprises a change in the sequence, length, symmetry, or a combination thereof in one or both mismatches and/or bulges of the pair of mismatches and/or bulges surrounding the second cleavage site.

14. The synthetic plant miRNA precursor of claim 13, wherein the modification comprises changing the nucleotide sequence of one or both mismatches and/or bulges.

15. The synthetic plant miRNA precursor of claim 13, wherein the modification comprises eliminating one or both of the mismatches and/or bulges.

16. The synthetic plant miRNA precursor of claim 13, wherein the modification comprises converting a nucleotide base pair between the two mismatches and/or bulges to a mismatch.

17. The synthetic plant miRNA precursor of claim 13, wherein the modification comprises deleting one or more nucleotides to convert one or more mismatches into a bulge.

18. The synthetic plant miRNA precursor of claim 1, wherein the modification comprises a change in the change in the sequence, length, symmetry, or a combination thereof in the series of mismatches and/or bulges between the second cleavage site and a third cleavage site.

19.-26. (canceled)

27. The synthetic plant miRNA precursor of claim 1, wherein the plant pest is an insect.

28. The synthetic plant miRNA precursor of claim 1, wherein the plant pest is a nematode.

29. The synthetic plant miRNA precursor of claim 1, wherein the plant pest is a fungus.

30.-34. (canceled)

35. An expression cassette or vector comprising a nucleotide sequence encoding the synthetic plant miRNA precursor of claim 1.

36. (canceled)

37. A method of producing a plant that is resistant to a plant pest, comprising
introducing into a plant or plant part the synthetic plant miRNA precursor molecule of claim 1 or the expression cassette or vector of claim 35, thereby producing a transgenic plant or plant part that is resistant to a plant pest.

38.-52. (canceled)


SEQUENCE LISTING (16 sequences)

SEQ ID NO: 1 - DNA, 224 bp, Artificial Sequence
  Synthetic oligonucleotide
gcgttattcggtgtttgaagcgtgctcattatctcctgtctgaaggggcctacggacggtgttgttccgctgctcgttcatggttccccatatctacttccatcatgttatagatctcgtctttggaagtagctttgggtttgcatgaccgaggagctgcaccacaccgtccgggcccgctctgacagaagagagtgagcacgcatcctaacacctgccattgt

SEQ ID NO: 2 - DNA, 226 bp, Artificial Sequence
  Synthetic oligonucleotide
gatgttttggatttcaagcgtgctcattatctcctgtctgaaggctccttctgaagggtcgttccgctgctcgttcatggttcccactatgctatctcatcatgtatgtctggagaagcgagattcttgagttaggcttgtggtttgcatgtccgaggagctgcagtgtcccctttgctggccgctctgacagaagagagtgagcacgcaccctgattttttatat

SEQ ID NO: 3 - DNA, 229 bp, Artificial Sequence
  Synthetic oligonucleotide
aataatcaccataatcgagtcgttgctcattatctcctgtctgaaggctccttctgaagggaacgttccgctgctcgttcatggttcccactatcagatcctcatcatgtagtaatgagaattttttttcgaggatctgtgtgtggtatacatgatcgaggagctttacgtacccaattccggagcactctgacagaagagagtgagcacgactagaaataaggaaacc

SEQ ID NO: 4 - DNA, 246 bp, Artificial Sequence
  Synthetic oligonucleotide
atttaaatagaccgatctcaggtgctcattatctcctgtctgaagggtcgtttcgcagggctggttccgctgctcgttcatggattccactaacctatctcatcatattgctatatgtctaatatgggctaataactagccctgaattgagataggattgtggtttacatgatcgagaagcagtaccgcccaagtgtagtccaatctgacagaagagagtgagcacgtaagtaactgtcaagattc

SEQ ID NO: 5 - DNA, 246 bp, Artificial Sequence
  Synthetic oligonucleotide
ttcttagaaacatgtatgtgtgtgctcattatctcctgtctgaagggtcgtttcgctgggttggttccgctgctcgttcatggttaccacaaacctatctcatcatattaatatatgtactttattcggaacaatattccgatgtattgagataggagggtggaatacatgatcgaggagctttaccacccttgtgtagtccaatctgacagaagagagtgagcacgctcagatcttgtaaactta

SEQ ID NO: 6 - DNA, 185 bp, Artificial Sequence
  Synthetic oligonucleotide
acgtttgctcattatctcctgtctgaagggccaaagcagtgaagagtttccgctgctcgttcatggttcctgttgagctatctcatcatgttatagatctcgttgagatagctatagaccgcatggctggggagctggaacccttcccctgatggccgatctgacagaagagagtgagcacgcat

SEQ ID NO: 7 - DNA, 188 bp, Artificial Sequence
  Synthetic oligonucleotide
acgtttgctcattatctcctgtctgaagggccaaagcagtgaagagtttccgctgctcgttcatggttcctgttgagctatctcatcatgttatagatctcgtctttgagatagctatagaccgcatggctggggagctggaacccttcccctgatggccgatctgacagaagagagtgagcacgcat

SEQ ID NO: 8 - DNA, 190 bp, Artificial Sequence
  Synthetic oligonucleotide
acgtttgctcattatctcctgtctgaagggccaaagcagtgaggagtttccgctgctcgttcatggttcctgttgagctatctcatcatgttatagatctcgtctttgagatagcaatatagaccgcatggctggggagctgtaacccttcccctgatggccgatctgacagaagagagtgagcacgcat

SEQ ID NO: 9 - DNA, 188 bp, Artificial Sequence
  Synthetic oligonucleotide
acgtttgctcattatctcctgtctgaagggccaaagcagtgaagagtttccgctgctcgttcatggttcctgttgagctatctcatcatgttatagatctcgtctttgagatagctatagaccgcatggctggggagctggtacccttcccctgatggccgatctgacagaagagagtgagcacgcat

SEQ ID NO: 10 - DNA, 190 bp, Artificial Sequence
  Synthetic oligonucleotide
acgtttgctcattatctcctgtctgaagggccaaagcagtgaagagtttccgctgctcgttcatggttcctgttgagctatctcatcatgttatagatctcgtctttgagatagctatatagaccgcatggctggggagctggtacccttcccctgatggccgatctgacagaagagagtgagcacgcat

SEQ ID NO: 11 - DNA, 245 bp, Artificial Sequence
  Synthetic oligonucleotide
tcgatgctttggttttgaagcgtgctcattatctcctgtctgaagggacaatgcagtgggcaggtacggctgctggatcatgcaagcctgataccaaactcatcatgtctttttaactttaggaaccccgacccaatcagtcgtctttgggtttggcttcagggtaacatggcccaggagcaggaccgcccccttggtgtccgctctgacagaagagagtgagcaccatcctgagccacccctcc

SEQ ID NO: 12 - DNA, 224 bp, Artificial Sequence
  Synthetic oligonucleotide
tcgatgctttggttttgaagcggcgtgctcattatctcctgtctgaacggacaatgcagtgggcaggtacggctgctggatcatgcaagcctgataccaaactcatcatgttatagatctcgtctttgggtttggcttcagggaatcatggcccaggagcaggaccgcccccttggtgtcccctctgacagaagagagtgagcaccatcctgagccacccctcc

SEQ ID NO: 13 - RNA, 14 nt, Artificial Sequence
  Synthetic oligonucleotide
uguuauagaucucg

SEQ ID NO: 14 - RNA, 35 nt, Artificial Sequence
  Synthetic oligonucleotide
guucaucauguaguaaugagguauaauaaccgaac

SEQ ID NO: 15 - RNA, 53 nt, Artificial Sequence
  Synthetic oligonucleotide
cucaucauauuaauauauguacuuuauucggaacaauauuccgauguauugag

SEQ ID NO: 16 - RNA, 26 nt, Artificial Sequence
  Synthetic oligonucleotide
cucaucauguuauagaucucguugag